(19) United States
(12) Patent Application Publication
(10) Pub. No.: US 2025/0149152 A1
(43) Pub. Date: May 8, 2025

(54) DYNAMIC CONFIGURATION OF MEDICAL DEVICES AND SYSTEMS USING JURISDICTIONAL CONSTRAINTS FOR ALGORITHM SELECTION

(71) Applicant: Medtronic, Inc., Minneapolis, MN (US)

(72) Inventors: Matthew R. Yoder, Franklin, WI (US); Joel R. Lauer, Clearwater, MN (US); Gaurav Makin, North Oaks, MN (US); Amruta Paritosh Dixit, Maple Grove, MN (US)

(21) Appl. No.: 18/835,634

(22) PCT Filed: Feb. 26, 2023
(86) PCT No.: PCT/IB2023/051049

§ 371 Date: Aug. 2, 2024

Related U.S. Application Data
(60) Provisional application No. 63/267,776, filed on Feb. 9, 2022.

(51) Int. Cl. G16H 40/20; G16H 10/60

(52) U.S. Cl. CPC G16H 40/20; G16H 10/60

(57) ABSTRACT

This disclosure is directed to medical systems and techniques for dynamic configuration of medical devices. In one example, a method is configured to access a data structure comprising an algorithm for health event detection in patient data generated by at least one of a medical device of the patient or a personal device of the patient based on a usage scenario. An association of the algorithm and the usage scenario in the data structure indicates that use of the algorithm for the usage scenario complies with one or more jurisdictional requirements.

### FIELD

The disclosure relates generally to medical systems and, more particularly, medical systems configured to facilitate compliant use of algorithms for monitoring patient data and detecting changes in patient health and/or to deliver therapy to a patient.

### BACKGROUND

Medical systems may monitor various types of data of a patient or a group of patients for one or several purposes. Amongst the numerous examples, some medical systems may record measurements of a patient and their heart as indicia of cardiac health for that patient, which may be memorialized in raw data and/or processed data formats; as one example, electric signals representing cardiac activity over a period of time may be memorialized as a cardiac electrogram (EGM), and then processed into other indicia of the cardiac health of the patient. In some examples, the medical system may monitor the cardiac EGM to detect one or more types of arrhythmia, such as bradycardia, tachycardia, fibrillation, or asystole (e.g., caused by sinus pause or AV block).

Medical professionals may use the medical system on their patients for a number of reasons such as having the medical system record patient data for future use. Some medical professionals rely on that data to provide their patients with the best medical care. For various purposes, a medical professional may program the medical system to operate as desired, which may be in accordance with a certain algorithm, and calibrate medical system components to detect health events, deliver a therapy, and so forth. In some examples, the medical system may include one or more of an implantable medical device or a wearable device to collect various measurements used to detect changes in patient cardiac health. In some examples, an implantable or a wearable medical device may be configured with an algorithm for monitoring patient cardiac activity for cardiac events.

### SUMMARY

Medical systems and techniques as described herein dynamically configure medical devices for the provision of medical care to patients in various locations and circumstances. In any given geographic region, there are a number of scenarios in which a medical device may be used with the intention to obtain a health benefit for the patient. While a medical device may be approved for its intended use in one geographic region, the same medical device may be unlawful in a different geographic region. This results in a gap in the quality of the medical care provided to patients from different jurisdictions where a person in one jurisdiction lacks access to the same medical care that a person in another jurisdiction receives, and both will likely face obstacles traveling between jurisdictions, regardless of the travel's purpose.

To illustrate by way of example, it may the case that simply having an implanted medical device runs a person afoul of their local jurisdiction; concurrently, another person may suffer or die needlessly because the same implanted medical device has not been approved in their local jurisdiction. In the United States, a government agency, such as the Food and Drug Administration (FDA), may have approved the implanted medical device for public use, but the FDA lacks authority outside of the US, and the same medical device may be prohibited in countries across Europe. The present disclosure provides addition examples of usage scenarios that have a tendency to create obstacles for people simply because of where a person lives.

Similarly, a particular medical algorithm used by a device (e.g., including non-medical devices) may be approved in one jurisdiction, but not in another jurisdiction. In the other jurisdiction, a different algorithm, or different version of the same algorithm, may have been approved prompting a manufacturer/developer to either build different configurations of a same medical device/medical application or lose access to the other jurisdiction. Even if the manufacturer/developer is willing to build separately configured devices/applications and assume extra costs in terms of resources and capital, it will take some time for those devices/applications to be ready for public consumption, further delaying access for the people of the other jurisdiction. There are other ways for person to lose access on the basis of jurisdictional grounds as explained in the present disclosure.

The medical systems and techniques described herein represent a computing service-based solution for verifying the medical care a patient receives, for example, from their medical device. To operate with freedom from government entities, a computing system running a health monitoring service for patients with cardiac maladies who use their medical devices to diagnose/detect, treat, and otherwise monitor patient activity must verify that service operations comply with applicable (government) authority (e.g., an agency similar to the FDA).

The medical systems and techniques may accomplish at least a proximate improvement in patient physical/mental health, especially considering that the above-mentioned issues can result in less efficacious medical care for the patient. In view of the above, the present disclosure describes a technological improvement or a technical solution that is integrated into a practical application.

In one example, a medical system comprises communication circuitry communicatively coupled to one or more devices comprising at least one of a medical device or another device of a patient; and processing circuitry configured to: access a data structure comprising an algorithm for health event detection in patient data generated by at least one of a medical device of the patient or a personal device of the patient based on a usage scenario, wherein association of the algorithm and the usage scenario in the data structure indicates that use of the algorithm for the usage scenario complies with one or more jurisdictional requirements; in response to the selection of the algorithm, generate information operative to perform an automatic configuration of the patient data to be interoperable with logic implementing the algorithm; and apply the selected algorithm to the automatically configured data.

In one example, a method by processing circuitry of a medical system comprises responsive to receiving a service request for a medical device, determining data indicative of one or more jurisdictional requirements corresponding to a patient location, wherein the service request comprises data identifying an algorithm for health event detection in patient data generated by the medical device or another device, in accordance with the service request, accessing structured data comprising an approved usage scenario for the algorithm to determine whether the algorithm complies with the one or more jurisdictional requirements, based on a determination that the algorithm complies with the one or more jurisdictional requirements, automatically configuring the patient data to be interoperable with the algorithm, and generating health event detection results based on an application of the algorithm to the configured patient data.

In another example, a second method by processing circuitry of a medical system comprises accessing structured data comprising an algorithm for health event detection in patient data generated by at least one of a medical device of the patient or another device of the patient based on a usage scenario, wherein association of the algorithm and the usage scenario in the data structure indicates that use of the algorithm for the usage scenario complies with one or more jurisdictional requirements, in response to the selection of the algorithm, generating information operative to perform an automatic configuration of the patient data to be interoperable with logic implementing the algorithm, and applying the selected algorithm to the automatically configured data.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates example environment of an example medical system in conjunction with a patient, in accordance with one or more examples of the present disclosure.

FIG. 2 is a block diagram illustrating an example configuration of a medical device, in accordance with one or more examples of the present disclosure.

FIG. 3 is a block diagram illustrating an example configuration of the external device of FIG. 1, in accordance with one or more examples of the present disclosure.

FIG. 4 is a block diagram illustrating an example architecture of the monitoring service of FIG. 1 that is operative on the computing system of FIG. 1, which may be coupled to the medical device and external device of FIGS. 1-4, in accordance with one or more examples of the present disclosure.

FIG. 5 is a flow diagram illustrating an example operation for administering medical care, in accordance with one or more examples of the present disclosure.

FIG. 6 is a diagram illustrating one flow for generating a device profile and another flow for referencing the device profile to configure a medical device to be interoperable with an algorithm version, in accordance with one or more examples of the present disclosure.

FIG. 7 is a diagram of a database structure for the device profile of FIG. 6, in accordance with one or more examples of the present disclosure.

Like reference characters denote like elements throughout the description and figures.

### DETAILED DESCRIPTION

In general, medical systems according to this disclosure implement techniques for configuring the system with approved functionality for a corresponding jurisdiction. The present disclosure describes a monitoring service as a computing service running on a computing system that communicates with a number of devices (e.g., patient devices including medical devices) over a network. The present disclosure further describes the monitoring service as a computing service for enforcing jurisdictional requirements and ensuring that the medical system complies with law and regulations governing a location of a patient. As an option, the patient may request an automatic configuration of a new algorithm enabling new functionality/enhancing current functionality of the medical system.

In general, one or more computing systems provide a number of monitoring services to patients—including those with medical devices (e.g., implanted and/or wearable medical device)—that, by way of performing a number of operations, monitor patient activity and detect changes in patient health (if any). As further described herein, the “monitoring service” (which may also be known as “health monitoring service” or simply “service”) generally operates in furtherance of medical care administered to a patient. The present disclosure describes “medical care” as referring to any product and/or process capable of making a patient more healthful and, typically, includes medical procedures, clinical evaluations, methods for providing therapy, methods involving diagnosis and/or treatment, healthcare-related processes, and/or the like.

Those within a medical industry, including device manufacturers, constantly improve upon their previous innovations in related technologies. Some are rapidly developing, testing, and deploying algorithms (e.g., machine learning algorithms) that are more efficient/effective. As described herein, each distinguishable algorithm may be assigned a unique identity amongst a plurality of algorithms; in some examples, the plurality includes different versions of a particular algorithm where each version qualifies as an independent algorithm. Each algorithm (e.g., version) requires separate validation by a relevant authority of a jurisdiction. Each algorithm (e.g., version) requires compatibility with at least one possible device configuration. As these algorithms become more popular, more are implemented for various usage scenarios, resulting in a patchwork of algorithms that have been validated by different jurisdictions.

The present disclosure further describes the monitoring service as being configured for verifying an algorithm that has been selected for patient data generated by a medical device. Confirmation or rejection may be based on an approval of an intended use or purpose (i.e., usage scenario as defined herein) of the algorithm and/or the medical device once programmed to run the algorithm. Approval, in general, may be granted based on compatibility between the patient data generated by the medical device and the algorithm. The monitoring service may determine that the medical device is properly configured to generate appropriate input/output to/from logic for executing the algorithm and therefore, approve of an application of the algorithm to the patient data. For instance, approval may be granted if programming within the medical device is compatible with the logic for executing the algorithm. Approval may require the patient data to be properly configured for the application of the algorithm. Approval may further require that the medical device (e.g., and its programming) conform its configuration to (e.g., characteristics of) the algorithm, otherwise the medical device cannot implement the algorithm nor run properly. If approval cannot be conferred to the medical device for not being appropriately configured, the monitoring service determines modifications to the medical device configuration and either simulates those modifications with the patient or provides the medical device with instructions on applying those modifications. Approval may further require that the algorithm comply with any applicable jurisdictional requirement associated with the medical device. Hence, approval may depend on satisfaction of one or more requirements, such as compatibility, compliance, among others.

Information describing a current configuration of a medical device and/or its patient data may refer to programming hardware/software components to be interoperable with logic for executing a current algorithm. The current configuration may require one or more modifications to conform to an appropriate configuration of a new algorithm selected for the patient. The present disclosure describes how to modify the current configuration of the patient data generated by the medical device or the medical device itself. One example of the modified configuration may indicate the same one or more input parameters to the algorithm, the same one or more output parameters, and/or other attributes that match the algorithm characteristics. A number of configurations may be pre-defined for similar algorithms. The monitoring service may select for patient data and/or the medical device the pre-defined configuration that best conforms to the algorithm characteristics.

The monitoring service may verify interoperability and validity of an algorithm with patient data generated by a medical device or the device itself. The monitoring service may evaluate the algorithm for the medical device under jurisdictional requirements for validation (“validation requirements”) which are established by some authority (e.g., a regulatory body) and interoperability (“interoperability requirements) which are dictated by device configuration. These validation requirements are conditions set forth during a validation process for the medical device and primarily applied to attribute information of a planned/intended usage scenario corresponding to the medical device. The interoperability requirements are restrictions placed on datasets of patient data of which at least some result from comparing a current device configuration with algorithm characteristics.

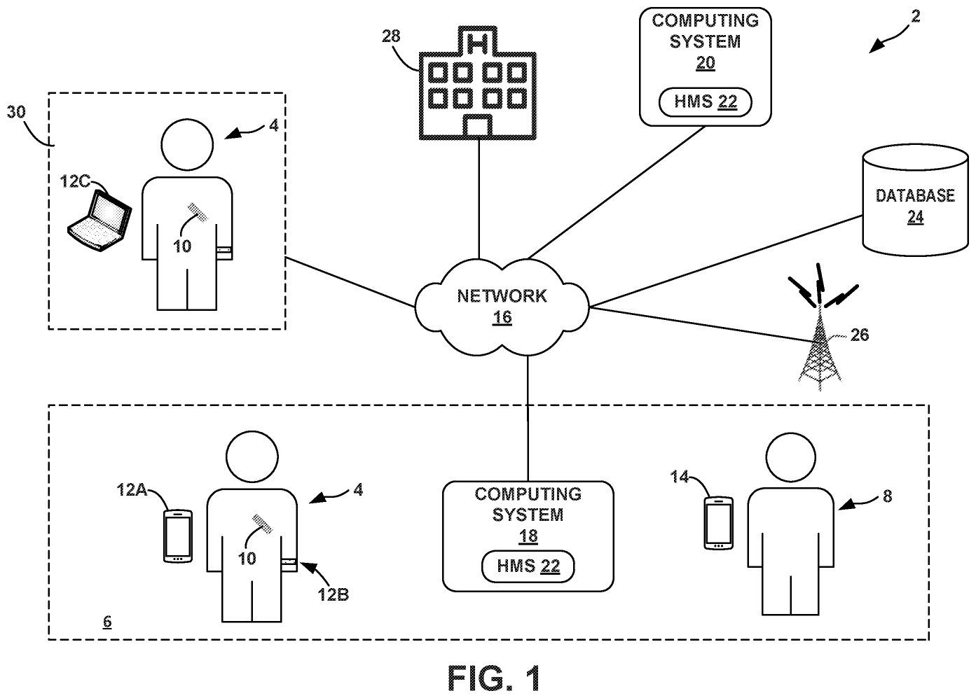

In general, the present disclosure represents “usage scenario” as an attribute dataset defining a set of circumstances (e.g., country, clinic type, algorithm/model version, and/or the like) in which a patient (e.g., patient 4 of FIG. 1) of a given medical device receives health event monitoring/detection and generates patient data to that effect. The usage scenario may also identify a particular algorithm/model version for application to the patient data. As described further below, the algorithm may correspond to a number of characteristics of which at least some identify one or more input parameters to the algorithm, one or more output parameters, and/or the like. Hardware/software components for enabling medical device operation may be compatible with the algorithm, for example, by enabling the number of characteristics associated with the algorithm.

The monitoring service may determine that the usage scenario satisfies the validation requirements, for example, if a usage scenario attribute indicates an algorithm/model identifier for an approved machine learning algorithm/model. The monitoring service may determine that the algorithm characteristics satisfy the interoperability requirements, for example, if the patient data is compatible (e.g., accessible) with programming for the application of the algorithm on the patient data. Assuming satisfaction of all requirements, the monitoring service may permit the application of the particular algorithm/model version. Otherwise, the monitoring service may reject the verification request (in part) because the model attribute does not comply with a validation requirement and/or an interoperability requirement. That is, the particular algorithm/model version may be incompatible with the patient data; while the monitoring service may refuse to apply the particular algorithm/model version based on that incompatibility, the monitoring service may exercise its option to override the rejection, thereby applying the particular algorithm/model version to the patient data. An override may be authorized if the monitoring service successfully pre-processes the patient data to be compatible with the particular algorithm/model version. In some examples, the monitoring service may automatically select or configure aspects of the algorithm, patient data, or device performance to meet the validation requirement.

The patient may have a patient device (e.g., a mobile device) in addition to their medical device to perform the above one or more operations on behalf of the patient to their benefit. The patient device may execute and run a monitoring application for coordinating re-configurations of the medical device and/or patient data when necessary (e.g., to implement an algorithm or configuration approved by local laws and regulations). When the patient device is connected to the monitoring service by way of a communicative coupling, the patient device may communicate messages on behalf of the medical device or another device configured for heath event monitoring. The patient device may communicate, in a message, a service request for the monitoring service to perform one or more operations. As one example service request, the patient device may communicate a message requesting verification of the algorithm and (if needed) an automatic configuration of the patient data for compatibility with the medical device and any algorithm executed by the medical device. As another example service request, the patient device may communicate a message requesting an algorithm that is compliant with one or more jurisdictional requirements. As another example service request, the patient device may communicate a message requesting an algorithm that is compatible with the patient data (as input). In one example, this request may authorize an application of the compliant and/or compatible algorithm to input patient data for generating health event detection results. The input patient data may be stored as raw device output or as processed results in structured data and one or both may be compatible for an interface with the algorithm.

The patient device may communicate a message requesting the monitoring service perform a patient assessment. The patient device may relay to the monitoring service patient data submissions by the medical device (e.g., based on interrogation data from the medical device). This message may request adjudication of an initial determination (e.g., initial detection or rejection) by the medical device. However, there may be a number of instances where the medical device is inhibited or unable to perform device functionality. For example, the patient device may impose hardware/software restrictions preventing the medical device from operating as a response to non-compliant/incompatible algorithms. Even when the medical device remains active/operational (e.g., running in the background), the monitoring application of the patient device may maintain an up-to-date copy of patient data by collecting required (e.g., recent) information (e.g., sensed physiological information, device interrogation data, and/or the like) in preparation for the patient assessment results from the monitoring service.

FIG. 1 is a block diagram illustrating an example system 2 configured detect health events of a patient 4, and to respond to such detection, in accordance with one or more techniques of this disclosure. As used herein, the terms “detect,” “detection,” and the like may refer to detection of a health event presently (at the time the data is collected) being experienced by patient 4, as well as detection based on the data that the condition of patient 4 is such that they have a suprathreshold likelihood of experiencing the health event within a particular timeframe. Examples of health events are a cardiac arrest, a ventricular fibrillation, a ventricular tachycardia, a stroke, a seizure, or a fall. The example techniques may be used with one or more patient sensing devices, e.g., IMD 10, which may be in wireless communication with one or more patient computing devices, e.g., patient computing devices 12A, 12B, and 12C (collectively, “patient computing devices 12”). Although not illustrated in FIG. 1, IMD 10 include electrodes and other sensors to sense physiological signals of patient 4, and may collect and store sensed physiological data based on the signals and detect episodes based on the data.

IMD 10 may be implanted outside of a thoracic cavity of patient 4 (e.g., subcutaneously in the pectoral location illustrated in FIG. 1). In some other examples, IMD 10 may be implanted at other subcutaneous locations on patient 108 such as at an abdominal location. IMD 10 may be implanted subcutaneously or submuscularly on the left midaxillary of patient 4, such that IMD 10 may be positioned on the left side of patient 4 above the ribcage.

IMD 10 may be positioned near the sternum near or just below the level of the heart of patient 4, e.g., at least partially within the cardiac silhouette. In some examples, IMD 10 takes the form of the LINQ™ ICM. Although described primarily in the context of examples in which IMD 10 takes the form of an ICM, the techniques of this disclosure may be implemented in systems including any one or more implantable or external medical devices, including monitors, pacemakers, defibrillators, wearable external defibrillators, neurostimulators, or drug pumps. Furthermore, although described primarily in the context of examples including a single implanted patient sensing device, in some examples a system includes one or more patient sensing devices, which may be implanted within patient 4 or external to (e.g., worn by) patient 4.

Patient computing device 12 (or simply “computing device 12”) may communicate with other devices, including IMD 10, via near-field communication technologies (e.g., inductive coupling, NFC or other communication technologies operable at ranges less than 10-20 cm) and far-field communication technologies (e.g., radiofrequency (RF) telemetry according to the 802.11 or Bluetooth® specification sets, or other communication technologies operable at ranges greater than near-field communication technologies). Computing devices 12 may communicate with IMD 10 and/or each other according to the Bluetooth® or Bluetooth® Low Energy (BLE) protocols, as examples. In some examples, only one of computing devices 12, e.g., computing device 12A, is configured for communication with IMD 10, e.g., due to operation of hardware (e.g., as part of a BLE receiver/transmitter module) and/or due to execution of software (e.g., part of an application as described herein) enabling communication and interaction with an IMD 10.

In some examples, computing device(s) 12, e.g., wearable computing device 12B in the example illustrated by FIG. 1, may include electrodes and other sensors to sense physiological signals of patient 4, and may collect and store physiological information and detect episodes and other health events based on such signals. Examples of the types of data managed by computing device(s) 12 may include sensed data, e.g., values of physiological parameters measured by IMD 10 and, in some cases one or more of computing devices 12, data regarding episodes of arrhythmia or other health events detected by IMD 10 and computing device(s) 12, and other physiological signals or data recorded by IMD 10 and/or computing device(s) 12.

In some examples, computing devices 12 take the form of personal computing devices of patient 4. For example, computing device 12A may take the form of a smartphone of patient 4, and computing device 12B may take the form of a smartwatch or other smart apparel of patient 4. Computing device 12B may be incorporated into the apparel of patient 4, such as within clothing, shoes, eyeglasses, a watch or wristband, a hat, etc. In some examples, computing device 12B is a smartwatch or other accessory or peripheral for a smartphone computing device 12A. In some examples, computing devices 12 may be any computing device configured for wireless communication with IMD 10, such as a desktop, laptop, a notebook computer, tablet computer, workstation, one or more servers, cellular phone, personal digital assistant, or another computing device that may run an application that enables the computing device to interact with IMD 10. Computing devices 12 may include one or more sensors configured to capture other sensed physiological data from the one or more sensors.

Network 16 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 16 may include one or more networks administered by service providers, and may thus form part of a large-scale public network infrastructure, e.g., the Internet. Network 16 may provide computing devices and systems, such as those illustrated in FIG. 1, access to the Internet, and may provide a communication framework that allows the computing devices and systems to communicate with one another. In some examples, network 16 may include a private network that provides a communication framework that allows the computing devices and systems illustrated in FIG. 1 to communicate with each other, but isolates some of the data flows from devices external to the private network for security purposes. In some examples, the communications between the computing devices and systems illustrated in FIG. 1 are encrypted.

Computing devices 12 are configured for wireless communication with various devices including IMD 10, other computing devices 12, and one or more computing systems, e.g., computing systems 20A and 20B (collectively, “computing systems 20”) via network 16. Computing systems 20A and 20B may be respectively managed by manufacturers of IMD 10 and/or computing devices 12 to, for example, provide cloud storage and analysis of collected data, maintenance and software services, or other networked functionality for their respective devices and users thereof. Computing devices 12 retrieve various types of information including datasets of patient data, such as event data (e.g., health events and/or alerts), monitored medical device data, and sensed physiological information that was collected and stored by IMD 10. In some examples, a client for a (e.g., cloud) computing service, referred to herein as health management service (HMS) 22, may run as an application on IMD 10 and/or one or more computing devices 12.

Computing system 20A may comprise, or may be implemented by, the Medtronic Carelink™ Network, in some examples. In the example illustrated by FIG. 1, computing system 20A implements a monitoring service (HMS) 22, although in other examples, either of both of computing systems 20 may implement HMS 22. As will be described in greater detail below, HMS 22 of system 2 facilities detection of health events of patient 4 by ensuring only approved algorithms of patient 4's jurisdiction are used for analyzing patient 4's data. HMS 22 may be implemented as a computing service, such as a cloud service or a local server, that operates, directly, with any interoperable medical device, over network 16 or, indirectly, via a remote client application for the medical device. In some examples, IMD 10 qualifies as an interoperable medical device for HMS 22. HMS 22, as a computing service, provides computing resources over a network, including compute, storage, and network resources. A cloud service is an example embodiment that provides its clients with access to virtualized applications, desktops, platforms, and other services.

HMS 22 may implement functionality to enable an appropriate (re)configuration of patient data generated by a medical device such as IMD 10 or an appropriate (re)configuration of the medical device itself. While HMS 22, operating from computing devices in a single location or multiple locations, may communicate with several medical devices connected by network infrastructure, an entity managing HMS 22 may have responsibility over health event monitoring and detection operations at these connected medical devices. Although the administration and provision of medical care in general falls under the control of an authority (e.g., a government agency) for a local jurisdiction of any patient, responsibility for compliance may ultimately fall on the entity behind HMS 22. Accordingly, the entity has configured HMS 22 to ensure a given patient is receiving proper medical care, for example, by maintaining their private information in a secure environment, directing an analysis of the patient data as by a specific algorithm for health event monitoring and detection, and configuring a device such as a medical device to operate appropriately with respect to applicating the specific algorithm to analyze the patient data.

HMS 22 may expose, via an interface, functionality for handling service requests over network 16. HMS 22 may generate the interface to be accessible through a network connection and to be viewable as content (e.g., graphical content) on a display device. The interface may include content to represent available function calls for patients, clinicians, and other service users to invoke, for instance, where each function call may be invoked for handling a specific service request. For a generic service request, a generic function call may be configured to conform a provision of medical care by IMD 10, and system 2, to a standardized configuration for same or similar medical devices, as established by the local jurisdiction. Other parties (e.g., the patient or the patient's clinician) may submit service requests invoking functionality for conforming IMD 10's configuration (e.g., of its patent data) to match an approved device configuration established by the local jurisdiction.

Detection accuracy, data privacy, and device security exemplify some of areas currently being standardized into minimum validation requirements for any given jurisdiction. Among typical requirements that a given jurisdiction can establish, many currently focus their rule making authority on regulating data privacy. By enforcing current rules and promulgating new rules when needed, the local jurisdiction compels its medical facilities, clinics, and device manufacturers into protecting patient 4 from being identified by a third party (e.g., an adversary). A local jurisdiction may codify rules that set forth a minimum level of privacy, and any algorithm performing an operation that is likely to leak patient 4's data may invalidate that algorithm for public use. Any algorithm considered for validation must protect patient 4's sensitive information from disclosure and/or misappropriation, for example, by maintaining at least the minimum level of privacy.

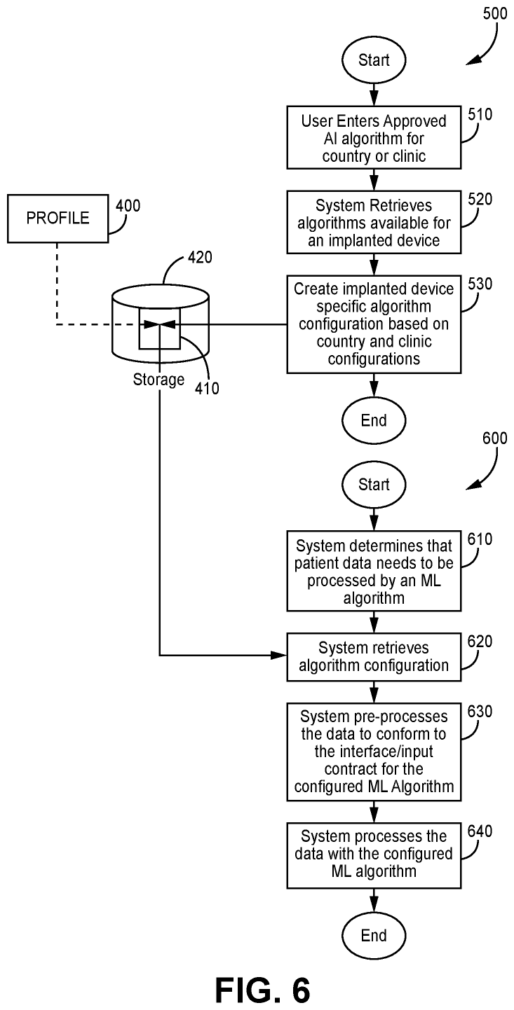

Handling a given service request, in general, may be automated by leveraging other functionality of HMS 22. In one example of the other functionality, HMS 22 may execute logic for handling service requests on behalf of patient 4 or clinician 8. The executed logic may include processor-executable instructions that, when executed (e.g., in memory), is configured to run a process for automatically invoking appropriate function calls when prompted by an incoming service request from a patient device. Further details are provided herein for FIG. 6 including a description of a number of examples of process 300 of FIG. 6. Examples of this process may be operative to direct hardware/software component(s) of HMS 22, in accordance with an identifiable algorithm for analyzing patient data to identify evidence of a health event that has occurred, is occurring, or will occur.

The present disclosure describes, in some examples, a service request for HMS 22 to handle where the service request may correspond to an example verification request. An example technique configured to properly handle the example verification request as an example process of a computing device may be programmed in logic for execution in a computing device. The logic, when executed in processing circuitry, causes the processing circuitry to verify a given algorithm is valid, for example, by determining whether the given algorithm complies with laws and regulations of a local jurisdiction, such as a governing jurisdiction for patient 4's current location. The same or similar technique may be used to validate IMD 10 based on the local jurisdiction's requirements. In general, the local jurisdiction may establish one or more validation requirements to set forth appropriate limits on using an algorithm with a computing device such as a patient device (e.g., computing device(s) 12, IMD 10 or another patient device) or a (server) device communicatively coupled to the patient device (e.g., for HMS 22 or another cloud service). The techniques described herein may include identification of a compliant algorithm and/or device configuration, and may ensure a proper configuration of any dataset generated by the medical device for implementing the algorithm.

A device located within the local jurisdiction is permitted to use only validated algorithms to evaluate patient 4's data for indicia of a particular health event (e.g., a cardiac event). As another validation requirement, the local jurisdiction may set forth rules to legitimize the device for an application of the algorithm, for example, by ensuring that the device is appropriately configured prior to approval to use the algorithm. Some of these rules, for instance, may require the device include components that are interoperable with logic for the algorithm. Assuming the algorithm uses a machine learning model, the local jurisdiction may require a specific version of a known model be used when implementing the algorithm in the device.

As described herein, the present disclosure codifies a usage scenario for IMD 10 and/or its patient data using a number of attributes of a dataset that describes how patient 4/user 8 intends to use a particular algorithm when analyzing the patient data. Example attributes identify a country or region (e.g., US or non-US country), a setting (e.g., clinical setting or public setting), an identity for the particular algorithm (e.g., a specific version of an algorithm for detecting cardiac events), and/or the like. While the particular algorithm may have been validated for certain uses in IMD 10, that validation is conditioned upon one or more jurisdictional requirements.

To effectuate the validation requirements set forth by the local jurisdiction, HMS 22 may run logic for simulating IMD 10 operation(s) in the cloud service devices. In a virtual embodiment of IMD 10, the logic may be a functional component of that embodiment. Accordingly, this logic component may define parameters associated with execution of an algorithm as described herein. Some example parameters that are defined in HMS 22 include input parameters including input features for a machine learning model. Some example parameters specify the machine learning model's components (e.g., a mathematical function or probability distribution). Some example parameters further define output parameters including (valid) output labels of the machine learning model. Modifying one or more of these parameters may cause a reconfiguration of patient datasets, resulting in reconfigured datasets that are compatible (e.g., as input to the algorithm). The logical component for virtualizing IMD 10 may also run the algorithm as a single logic component. In an alternative example, another program may be executed to run the algorithm as a separate logical component. The reconfiguration of the patient datasets allows interoperability between the separated logical components or between the logical component for the running algorithm and/or IMD 10 itself.

In another example where logic for running the algorithm is interoperable with IMD 10, certain device components of IMD 10 may be modified (e.g., reprogrammed) to conform to the validation requirements. For instance, IMD 10 may be configured with hardware/software components to execute a particular algorithm and an accompanying model. These components may include program code or logic that when executed, perform one or more device operations according to the algorithm and model. These components may be configured to define parameters for the program code or logic, including any input features, machine learning components (e.g., mathematical function or probability distribution), and/or output labels of the particular algorithm/model. Knowing these parameter definitions, HMS 22 may generate information for re-programming the device components to be compatible with a different algorithm/model. The generated information may identify modifications to the parameters such that the program code or logic of the device components may be modified to be interoperable with logic for implementing the different algorithm/model. HMS 22 may generate results data indicating a successful simulation of IMD 10 according to the intended use of IMD 10.

HMS 22 may maintain, in database 24 or another external location, an up-to-date list of approved algorithms (e.g., by version) available for use by IMD 10 and other medical devices (e.g., of a same type as IMD 10). Database 24 may implement a schema to define each algorithm in terms of a number of attributes where an algorithm version identifier serves as an index. Database 24 may use the schema to organize attribute information into separate profiles for each device type. An example profile for IMD 10 includes a list of approved algorithms corresponding to that device type. Some examples of attributes indicate which countries or jurisdictions have approved a particular algorithm version for use in a medical device and/or in a clinical setting. Examples of “clinical setting” may indicate specific clinics or other medical facilities, a particular clinical trial, and/or groups of patients enrolled in a clinical trial. Another attribute may identify each construct (e.g., machine learning model such as a neural network) that the particular algorithm version implements.

According to one example, where HMS 22 uses database 24 to ensure an algorithm is in a compliance with applicable laws and regulations. Consider an example where a specific algorithm is configured to determine whether a cardiac EGM sample is indicative of a cardiac episode (in general), patient 4 may have IMD 10 programmed with detection logic that implements the specific algorithm, configuring IMD 10 to detect cardiac episodes. HMS 22 may maintain information (e.g., records) in database 24 with attributes identifying each country/jurisdiction and/or device type in which the specific algorithm has been validated; for this reason, HMS 22 may determine whether the specific algorithm is currently approved for use in IMD 10 and/or with data generated by IMD 10. When patient 4 brings IMD 10 online with HMS 22 and prior to activating IMD 10 into service, IMD 10 may communicate a message to HMS 22 (and/or HMS 22 may retrieve information from IMD 10) having an algorithm version identifier and possibly additional attribute information for the specific algorithm. HMS 22 may receive the algorithm version identifier, identify the specific algorithm, and then, based on corresponding attribute information for the specific algorithm, respond to IMD 10 with a reply message indicating confirmation of the specific algorithm's verification.

HMS 22 may combine the above identifier for the specific algorithm with various attribute(s) for defining (at least part of) a usage scenario that has been approved for the specific algorithm. HMS 22 may store, in memory of computing system 20A, definition data for each approved usage scenario of the algorithm using a single attribute or a combination of two or more attributes from the following example attributes. In general, attribute information for the usage scenario characterize an intended use for the specific algorithm and correspond to satisfied jurisdictional requirements: Identifier(s) of a specific location (e.g., geographic region) where the intended use has been approved, such as country or jurisdiction name, clinic/facility name, and/or the like; identifier(s) for approved medical device(s), such as a serial number or a device model/type; identifier(s) for an approved setting/purpose of the intended use, such as a general public use or a clinical trial setting; and so forth. Instead of a numerical or alphanumerical value, an identifier may be a Boolean value, for example, indicating that a clinical trial setting is true or false.

In accordance with the present disclosure, a “jurisdiction” generally refers an area or territory representing an extent over which an entity exerts authority, and the “jurisdiction” for any given usage scenario may refer to an underlying geographic region for which medical systems are permitted to have implemented a specific algorithm for detecting health events in patient data. HMS 22 may determine that patient 4's intended use of the specific algorithm is legitimate for any person in any jurisdiction upon establishing satisfaction of one or more requirements therein. To illustrate by way of example, it may the case that simply having an implanted medical device runs a person afoul of a local jurisdiction; concurrently, another person may suffer or die needlessly because the same implanted medical device has not been approved in their local jurisdiction.

In the United States (US), a government agency, such as the Food and Drug Administration (FDA), may have approved an implanted medical device for public use, but the same medical device may be prohibited in countries across Europe. This is (in part) because the FDA can only govern the provision of medical care to the public of the United States and has no authority over the medical care being provided/authorized for public use in other jurisdictions; for at least this reason, the medical device's approval for public use is territorially limited to the jurisdiction of the United States. Obtaining global coverage would require approval in in every jurisdiction, and in many instances, a jurisdiction will enforce different rules causing the same medical device to be non-compliant in this jurisdiction and compliant in another jurisdiction. The above example introduces a usage scenario for the United States and the jurisdiction over which the nation governs. It should be noted that the present disclosure envisions medical device having additional scenarios, some of which can be generated by partitioning the public use into multiple categories (e.g., by age) and/or introducing new usage scenarios for non-public entities (e.g., government use, private university use, and so on).

Enforcing applicable laws and regulations of a jurisdiction/geographic region on the provision of such medical care often falls on the particular jurisdiction, specifically a designated governing body who promulgated the laws and regulations. The particular jurisdiction may grant authority to a relevant body (e.g., an agency), and that relevant authority may effectuate codification of their promulgated laws and regulations, setting forth a policy from which a medical device can be approved for public use; however, policy enforcement may be designated to a completely different agency. Often, this arrangement leads to several issues, a number of which negatively affect the medical care given to patients. Further, the need for developers of medical technologies to acquire approvals in numerous jurisdictions may create an administrative and logistical burden in their commercialization of such technologies.

Details regarding usage scenarios for the specific algorithm that may be applicable to one or more of the medical devices described herein are as follows. Data for codifying the specific algorithm may be arranged (e.g., structured) into attribute(s) of an example usage scenario. HMS 22 may configure attribute information of the example usage scenario to authorize the specific algorithm for a variety of devices, for example, by generating generic attribute information. The generic attribute information may be considered suitable (at the very least) by identifying the underlying geographic region of the jurisdiction. Some examples may define the generic attribute information for the example usage scenario as including a recognizable descriptor (e.g., formal name) as an identifier for the underlying geographic region.

It is appreciated that attributes defining approved uses of the specific algorithm can be articulated at multiple levels of specificity where each level confers a different usage scenario. To illustrate by way of example messages, IMD 10 may include, as a location attribute, a country abbreviation for the United States in an example message requesting verification/approval of the specific algorithm for general public use by patient 4 in the United States while another example message, by including an identity of a particular clinical trial, may request verification/approval for an experimental use in a clinical trial setting. While the experimental use request may imply that the specific algorithm has not yet been approved for general use, this may not necessarily be the case for there may other reasons to run experiments on the specific algorithm. Nonetheless, the attribute indicating the clinical trial setting may override the country restriction on the general public use of the specific algorithm in IMD 10.

In the example depicted by FIG. 1, patient 4 who, at present time, can be found within the US may have a medical device (e.g., IMD 10) or a computing device (e.g., computing device 12A) for monitoring various data for health events. One or both of the above devices may execute logic for the specific algorithm and commence such monitoring. Prior to authorizing the specific algorithm for one or more device types, patient 4 via computing device 12A may request confirmation from HMS 22 that the specific algorithm has been approved for patient 4's to use while located in the US. HMS 22 may access from computer memory attribute(s) of the example usage scenario and extract a location attribute for comparison with an identifier for the US as the underlying geographic region. In general, HMS 22 may confirm prior approval if the location attribute corresponds to the present jurisdiction of patient 4, which can be determined, for example, by accessing a data structure having the location attribute and comparing that attribute to a formal name of the underlying geographic region. HMS 22 may determine that the location attribute matches “US” and communicate a confirmation responsive to that determination.

HMS 22 may confirm approval in a number of other ways, for example, by verifying compliance by the specific algorithm for the jurisdiction of patient 4. In one example, because HMS 22 only authorizes an algorithm if that algorithm complies with applicable requirements of their jurisdiction, having the data structure in the computer memory signifies a previous confirmation by HMS 22 regarding the satisfaction of the jurisdiction requirements. In this manner, HMS 22 may rely on a presence of the data structure in the computer memory to confirm satisfaction of any jurisdictional requirement(s) of the underlying geographic region. HMS 22 configures the data structure in the computer memory to operate as an embodiment of the example usage scenario and by doing so, facilitates subsequent requests for confirmation. This is because approval for the intended use of the specific algorithm can be assumed by detecting the presence of the data structure in the computer memory.

HMS 22 generates a (device) profile or configuration to include attributes in accordance with the schema described herein. The profile of any medical device includes information defining the samples of training data to use in building the model for this device. The system tracks changes to a model's training samples as well as the model's predictive label. In addition to the input features and the output prediction, other monitored attributes for each device includes a device model or ID, an algorithm ID, a clinic/facility name, etc. A clinic may change models for a new clinical trial.

After determining that a given algorithm identified in a request from IMD 10 is now or has previously been approved for the requested usage scenario, HMS 22 may store a record of that verification is stored in database 24. If, according to one example, IMD 10 submits a request for an automatic configuration for an algorithm approved for use in a clinical setting, HMS 22 may store in database 24 a record having information corresponding to suitable logic and other instructions for implementing the approved algorithm and, as an option, any experiment for testing the approved algorithm. As one benefit, these records can be retrieved in a lookup operation in database 24 if IMD 10 or a similar medical device requests verification at some point in the future.

The schema described herein for database 24 may define additional or fewer attributes for records of approved algorithms and/or device profiles. As another possible attribute, database 24 may incorporate into the above schema information identifying a machine learning model being implemented by a specific algorithm. One example attribute may be a unique identifier (ID) for the machine learning model. This attribute may be incorporated into the schema as an index, for example, as a replacement for or in combination with an algorithm version attribute in the database 24 main table.

As noted above, IMD 10 may communicate additional data (e.g., attributes) in messages requesting verification services from HMS 22. One example attribute may identify IMD 10 (e.g., by device type), and based on this attribute, HMS 22 may retrieve a profile for IMD 10 (specifically) or its device type. An example device profile for IMD 10 may indicate which algorithms have been approved for use in that device. Therefore, in addition to location and/or setting, the usage scenario may be further fine-grained with a device type attribute such that medical devices similar to IMD 10 may have to be approved for use with the specific algorithm in order to be into service for patient 4.

One example function provided by HMS 22 as a verification service may enable (e.g., automatic) configuration of IMD 10 and/or IMD 10's data for implementing an algorithm after determining that the algorithm has been approved for a corresponding usage scenario. As described herein, the algorithm and its intended use may comply with applicable rules of its jurisdiction and thus, receive approval. As an alternative, the verification service may assume compliance or, as one option, rely on stored information, for example, in database 24. A device profile for IMD 10 and/or a record of a previous verification may indicate that the algorithm is an approved algorithm for patient 4. It should be noted that the previous verification of the algorithm may or may not be based on its previous usage scenario.

One example operation, an evaluation operation, includes an application of a machine learning model on input data (e.g., samples of cardiac EGM data) and a prediction by the model as output data (e.g., positive or negative arrhythmia detection). HMS 22 may direct the (re)programming of logic for implementing the evaluation operation regarding a possible occurrence of an arrhythmia in patient 4. With the logic for the approved algorithm, HMS 22 may program another logical component to match a configuration of the approved algorithm. Therefore, once properly configured, appropriate logic for the above evaluation operation should be configured to accept, as input arguments, one or more input samples and then, generate, as output, a value indicative of its prediction/determination when invoked for each sample.

Under certain conditions, any algorithm that has not been approved by the local jurisdiction or otherwise validated under the local jurisdiction's rules may still be used to evaluate data generated by IMD 10 while monitoring patient 4 for cardiac events. This may occur when the algorithm is determined to be incompatible with a dataset of patient data and therefore, is unable to access and/or process parameter values describing a physiology (e.g., a cardiac health) of patient 4. These conditions may qualify the algorithm for an override of normal validation requirements. HMS 22 may implement a particular type of service request for specifying certain validations requirement(s) to receive an override, allowing an application of the algorithm despite a lack of an approval by the local jurisdiction.

In another example, a different type of service request, which may be referred to as a configuration request, for specifying a certain schematic representation for data generated by a medical device for patient 4 such as IMD 10. This may occur when the algorithm is determined to be interoperable with a device executing it, which may be IMD 10, device(s) 12, or HMS 22. In one example, HMS 22 may configure/re-configure any patient data generated by the device to be compatible with the algorithm. To illustrate, HMS 22 may identify characteristics of the known model, including input features, model components, and/or output labels, and (re)configure the patient data for the known model. Configuring the patient data may enable logic for running the algorithm to retrieve (e.g., extract) the input features (e.g., as feature vectors or streams).

In another example, separate and independent types of service requests may be combined into yet another type of service request for submission to HMS 22. In one example, HMS 22 may override any jurisdictional requirement that cannot be satisfied. HMS 22 may have codified, in a software program, configuration logic for effectuating an appropriate configuration or reconfiguration of the patient data into properly configured patient data that is interoperable with the known model. The overridden jurisdictional requirement may affect the proper configuration/reconfiguration of the patient data. Although a requirement override may involve a reconfiguration of the patient data, in other examples, the reconfiguration of the patient data may not be necessary.

To further illustrate by way of another example validation requirement, the local jurisdiction may designate IMD 10 as safe for public use but with only approved algorithms in addition to validating/approving the algorithms themselves. Assuming an algorithm has not yet been validated according to the rules of the local jurisdiction, HMS 22 may not approve an algorithm for IMD 10 and/or any patient data generated by IMD 10 unless a medical professional (i.e., a clinician such as user 8) for patient 4 intends to use IMD 10 in a clinical setting. Given the clinician setting and oversight provided by clinician 8, HMS 22 may override the example validation requirement until the local jurisdiction classifies the algorithm as approved for use in IMD 10 for clinical trials and other experimental use cases. Otherwise, if the local jurisdiction has not yet determined that an algorithm causes IMD 10 to be reliable and effective, the local jurisdiction may not approve use of the algorithm in the jurisdiction and/or for the usage scenario.

Patient 4 desires seamless configuration (including re-configurations by a monitoring service such as HMS 22) of their data in an uninterrupted evaluation of that data by HMS 22 and/or IMD 10 for detecting cardiac events. IMD 10, having an intended purpose of detecting cardiac episodes in a time sequence of patient data samples, may operate assuming patient 4 is currently located in an approved country or region for using IMD 10 to run Algorithm A in a public setting and/or assuming HMS 22 is currently located in an approved country or region for running Algorithm A in a (simulated) public setting. If IMD 10 cannot be used for the evaluation by Algorithm A, HMS 22 may run the algorithm and perform the evaluation.

In another example, Algorithm B instead of Algorithm A may be approved for the jurisdiction of the current patient location. For this reason, HMS 22 may generate information for automatically configuring samples of device data to be compatible with Algorithm B. A logic component of HMS 22 may use this information to modify the device data into compatible input(s) for application of Algorithm B. Alternatively, the information may instruct IMD 10 to incorporate one or more changes to input features, model components (e.g., function parameters), and/or output labels such that device logic becomes interoperable with logic implementing Algorithm B. In one example, Algorithm B is a later version of Algorithm A. In yet another example, patient 4 may be in a clinical setting for testing Algorithm C. Algorithm A and Algorithm B are not being tested, requiring a reconfiguration of IMD 10 to be compatible with Algorithm C. Even though Algorithm C has not been approved in that country and/or for public use, the clinical setting overrides the validation requirement(s) behind the lack of approval, allowing patient 4 to opt-in the clinical trial.

There are other reasons for verifying an intended use of IMD 10 and other medical devices. Patient 4 may intend to use IMD 10 as prescribed by a medical professional, which typically involves using the device within manufacturer restrictions. Alternatively, a medical professional may have prescribed patient 4 an off-label use of IMD 10, thereby deviating from the intended purpose of the medical device. In the local jurisdiction described above, an agency having relevant authority over validation may have approved one usage scenario of a particular algorithm, but the same agency may not have approved another usage scenario, such as one indicating an off-label intended use, for the same particular algorithm.

The medical systems and techniques create hardware/software components to improve the efficiency of assessing patients with implantable medical devices. In some examples, computing system 20A, on behalf of and for HMS 22 to use as input samples, may retrieve/store cardiac EGM data that IMD 10 has (e.g., recently) recorded. HMS 22 may direct hardware/software components of computing system(s) 20 in pre-processing the cardiac EGM data to conform to an interface/input contract for the approved algorithm. HMS 22 may configure one or more input sources to feed the cardiac EGM data into memory that is arranged into an input feed for the logic configured for the approved algorithm. Upon execution, the logic may find accessible the input feed, run the approved algorithm, and generate output indicative of any detected cardiac episodes. Alternatively, HMS 22 may return the pre-processed data back to IMD 10 for device logic to perform the approved algorithm.

For example, computing system 20A may pre-process the cardiac EGM data, which may reflect a single input source or multiple input sources (e.g., leads), by normalization, selection, filtering, among a number of other transformations. Computing system 20A may pre-process the cardiac EGM data into a series of samples where each is of a specific sample length. If the cardiac EGM data is multi-dimensional where each dimension reflects a lead, computing system 20A may pre-process the cardiac EGM data by removing one or more dimensions and/or selecting a single dimension to create one-dimensional pre-processed cardiac EGM data. As another example, computing system 20A may pre-process the cardiac EGM data into one or more features (e.g., values) per sample. If the algorithm is a machine learning algorithm, computing system 20A may feed, as input, the one or more features into a machine learning model, which generates, as output, a determination regarding whether the one or more features are indicative of a cardiac episode.

Patient 4 and/or clinician 8 may have programmed IMD 10 and/or computing devices 12A, 12B, or 12C to upload (at regular intervals) datasets of patient data sensed or measured from patient 4 over a time period. Clinician 8 may enter data for a message requesting algorithm verification/automatic configuration. The data may be used in a service request sent to HMS 22 via a network connection facilitated by computing system 20 for running HMS 22 as a cloud service.

Area 6 may be where a type of medical facility (e.g., a clinic) is located, for example, to facilitate HMS 22. Under direction of HMS 22, clinician 8 may leverage this monitoring service to benefit different patients, including patient 4 and other patients. Medical systems may have facilities such as hospital 28 and other medical facilities for both clinical and non-clinical health monitoring. Patient 4 may receive medical care while at a non-medical facility 30, such as their home, office, or retirement community, while clinician 8 submits patient 4's medical device data for evaluation by an approved algorithm or an experimental algorithm.

Clinician device 14, a computing device similar to devices 12, may be communicatively coupled to hospital 28 and non-medical facility 30 by a network connection maintained, in part, by computing system 18. Any data of patient 4 recorded and/or stored at hospital 28 and non-medical facility 30 is immediately copied to computing system 20 for HMS 22 to handle. Clinician 8 may enter data to identify a particular algorithm for IMD 10 such as a unique identifier for distinguishing the algorithm from other algorithms including different version of the particular algorithms. Patient 4 may have programmed IMD 10 or any one or more of devices 12 to transmit, via a network connection, sequential samples of patient data sensed or measured from patient 4.

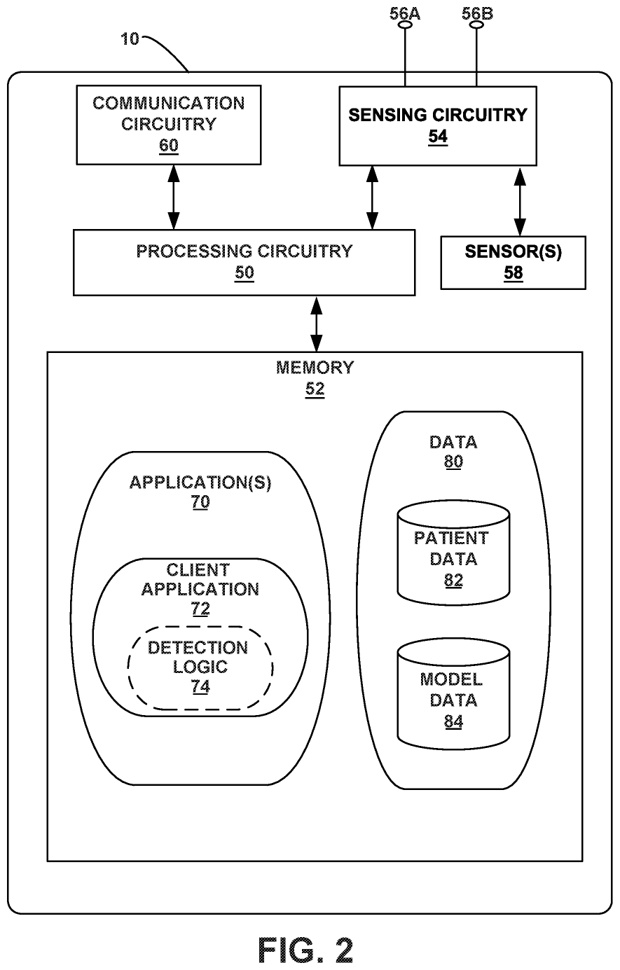

FIG. 2 is a block diagram illustrating an example configuration of IMD 10 of FIG. 1. Similar to system 2 of FIG. 1, IMD 10 forms part of a medical system. Some embodiments of the medical system couple IMD 10 with a (e.g., remote or local) computing system configured to run one or more computing services including a monitoring service. A computing service may provide additional storage, processing, and network resources to IMD 10 and/or an external device coupled to IMD 10 such as computing device 12A of FIG. 1. At least one application may be executed and running in either IMD 10 or the external device as a client or an agent of at least one computing service such as the monitoring service (HMS). In FIG. 1, HMS 22, an example of the monitoring service, may be configured to support IMD 10.

As shown in FIG. 2, IMD 10 includes processing circuitry 50, memory 52, sensing circuitry 54 coupled to electrodes 56A and 56B (hereinafter, “electrodes 56”) and one or more sensor(s) 58, and communication circuitry 60. Processing circuitry 50 may include fixed function circuitry and/or programmable processing circuitry. Processing circuitry 50 may include any one or more of a microprocessor, a controller, a graphics processing unit (GPU), a tensor processing unit (TPU), a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 50 herein may be embodied as software, firmware, hardware, or any combination thereof. In some examples, memory 52 includes computer-readable instructions that, when executed by processing circuitry 50, cause IMD 10 and processing circuitry 50 to perform various functions attributed herein to IMD 10 and processing circuitry 50. Memory 52 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Sensing circuitry 54 may monitor signals from electrodes 56 in order to, for example, monitor electrical activity of a heart of patient 4 and produce ECG data for patient 4. In some examples, processing circuitry 50 may identify features of the sensed ECG, such as heart rate, heart rate variability, intra-beat intervals, and/or ECG morphologic features, to detect an episode of cardiac arrhythmia of patient 4. Processing circuitry 50 may store the digitized ECG and features of the ECG used to detect the arrhythmia episode in memory 52 as episode data for the detected arrhythmia episode.

In some examples, sensing circuitry 54 measures impedance, e.g., of tissue proximate to IMD 10, via electrodes 56. The measured impedance may vary based on respiration and a degree of perfusion or edema. Processing circuitry 50 may determine physiological data relating to respiration, perfusion, and/or edema based on the measured impedance.

In some examples, IMD 10 includes one or more sensors 58, such as one or more accelerometers, microphones, optical sensors, temperature sensors, and/or pressure sensors. In some examples, sensing circuitry 54 may include one or more filters and amplifiers for filtering and amplifying signals received from one or more of electrodes 56 and/or sensors 58. In some examples, sensing circuitry 54 and/or processing circuitry 50 may include a rectifier, filter and/or amplifier, a sense amplifier, comparator, and/or analog-to-digital converter. Processing circuitry 50 may determine physiological data, e.g., values of physiological parameters of patient 4, based on signals from sensors 58, which may be stored in memory 52.

Memory 52 may store applications 70 executable by processing circuitry 50, and data 80. Application(s) 70 may include client application 72 and, as an option, detection logic 74. Processing circuitry 50 may execute detection logic 74 for general health monitoring and health event surveillance which, in some instances, may result in detecting a health event for patient 4. The detection may be based on combination of one or more of the types of data (e.g., sensed data including sensed cardiac activity and other physiological data as described herein), which may be stored as patient data 82. In some examples, patient data 82 may additionally include data sensed by other devices, e.g., computing device(s) 12, and received via communication circuitry 60.

Client application 72, operating as a client of a computing service (e.g., HMS 22 of FIG. 1) accessible over a network connection, may communicate data, for example, patient data 82 over a network to an external computing system for the computing service. As described herein, the communication may initiate an application of a machine learning model to model data 84. Client application 72 (and possibly other application(s) 70) may be configured to direct operations of IMD 10 including any input/output functionality.

Client application 72 may be run (as an executable) to initiate a set of actions that may be laid out as processor-executable instructions in a computer file. In general, client application 72 may be operative to determine whether patient data 82 is indicative of a health event. The running executable of client application 72 instructs processing circuitry 50 to start specific computer processes, such as an instance of an algorithm for patient assessment as described herein. When embodied in a medical device such as IMD 10, client application 72 may activate programmable logic, for example, to generate model data 84 from one or more samples of patient data 82, evaluate model data 84 with a machine learning model and algorithm, and then, generate output indicative of a possible occurrence during the one or more samples.

In one example, detection logic 74 may be stored in memory 52 as programmable logic (in a computer program) for enabling a patient assessment to detect any health events. In one example, detection logic 74 may be operative within IMD 10 as logic circuitry to facilitate the patient assessment, for example, by applying an algorithm to one or more samples of patient data 82 and determining whether a health event occurred. The algorithm generally implements rules that may be programmed into memory 52 (e.g. as an executable) for detection logic 74. In addition to controlling the evaluation of patient data 82, these rules may control input data and output data of detection logic 74. In some examples, the above-mentioned algorithm, which may be characterized as a machine learning algorithm, implements rules corresponding to one or more models, such as neural networks, decision trees, and/or thresholds.

Client application 72 may invoke functionality of detection logic 74 when performing the patient assessment, for example, by applying a machine learning model to patient data 82. Model data 84 may store input features for initiating the application of the machine learning model, and at least one function call of detection logic 74 may be used to generate model data 84 from patient data 82.

Detection logic 74 may include software code for generating an interface (e.g., as an executable) through which relevant algorithmic functionality is called when evaluating patient data 82 for evidence of a health event. When detection logic 74 detects a possible health event, client application 72 may direct IMD 10 to generate an alert, for example, by producing an alarm as output. The health event detection may further prompt IMD 10 to store, in data 80, each sample of patient data 82 that caused the detection (and in some cases a window of data preceding and/or following the detection).

In some examples, the algorithm implemented by detection logic 74 may configure IMD 10 to detect cardiac arrhythmias, worsening heart failure, or other health events based on cardiac activity data as recorded in an ECG/EGM and/or other physiological data indicating the electrical or mechanical activity of heart of patient 4 (FIG. 1). In some examples, detection logic 74 may detect stroke based on such cardiac activity data. In some examples, sensing circuitry 54 may detect brain activity data, e.g., an electroencephalogram (EEG) via electrodes 56, and detection logic 74 may detect stroke or a seizure based on the brain activity alone, or in combination with cardiac activity data or other physiological data.

As described herein, IMD 10, and by extension HMS 22 of FIG. 1, should follow local jurisdictional laws and regulations, for example, by limiting application(s) 70 to approved device functionality. By submitting and then, obtaining market approval from a relevant authority for the algorithm used in IMD 10, for example, for identifying evidence of cardiac episodes in samples of cardiac activity data, IMD 10 may be configured to provide its patients with medical care only if the relevant authority has reviewed the underlying operations behind the device functionality and declared them safe for patient use.

It should be noted that while IMD 10 may be approved for public use, for instance, because the above relevant authority validated the algorithm programmed into detection logic 74, the relevant authority (as an option) may set forth a number of validation requirements for IMD 10 to follow. Assuming the relevant authority covers a particular geographic area (e.g., a country, a region, or a group of countries/regions), HMS may be configured to enforce the validation requirements described herein on IMD 10 such that the algorithm used for detecting cardiac episodes always is compliant with the particular geographic area as well as other geographic areas (e.g., other countries). However, HMS may override the corresponding validation requirement for approved clinical trials within the particular geographic area; an override may acceptable if the relevant authority approved of one or more exceptions to the validation requirements such as when specific facilities in the particular geographic area provide at least one clinical setting for running a trial where a clinician observes IMD 10 operation with an unapproved algorithm implemented in detection logic 74; in these examples. In this manner, IMD 10 is configured to refrain from operating without at least some approval of its detection logic 74 even if the algorithm implemented in that logic has not been formally validated.

HMS 22 may be accessible to medical devices such as IMD 10 via a network service through which messages (e.g., requests, responses/replies, and/or the like) are exchanged. Some messages provide attribute information identifying which algorithm IMD 10 is using for detection logic 74. Such messages may be informative while other messages may be requests for approval of a proposed/desired algorithm for detection logic 74. In response to the other messages, HMS 22 may return reply messages indicating either approval or disapproval of the proposed algorithm. HMS 22 may return a reply message indicating approval of detection logic 74 upon determining that the proposed algorithm complies with a country's regulations, a clinic's trial, or another acceptable usage scenario. In this manner, HMS can ensure IMD 10 only uses approved logic for detecting cardiac episodes.

A message may describe a usage scenario using attributes indicative of a current patient location and communicated to a local/remote device of a monitoring service, such as HMS 22 of FIG. 1, prompting a response indicating approval or disapproval of the requested algorithm. Client application 72 may be configured to automatically transmit patient data 82 in response to an approval from HMS 22. Client application 72 may prepare each input (e.g., sample) feed by processing datasets of patient data 82, in some examples, after obtaining approval of an algorithm that is to be used for some of the example rules of detection logic 74.

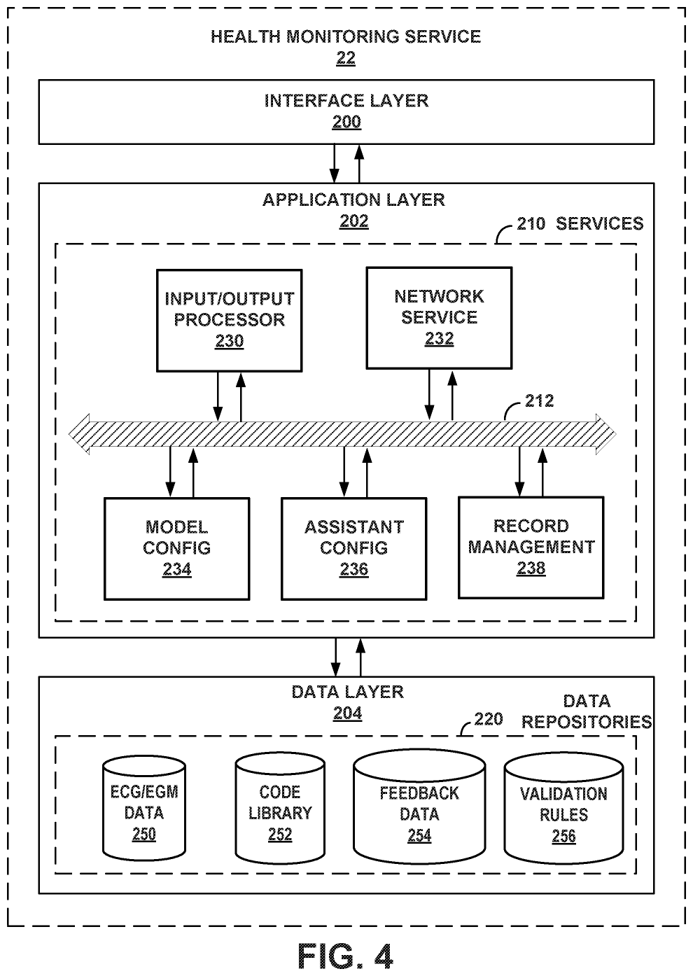

As described in further detail for FIG. 4, HMS 22 may receive requests for services including services that provide application resources and functionality. For example, HMS 22 may receive a message requesting verification of a proposed/desired algorithm for use in a particular jurisdiction, requesting configuration of patient data for input to the proposed/desired algorithm, and further requesting that HMS 22 perform the proposed application on (raw) input samples provided by IMD 10 or on (refined) input parameters computed from the input samples. The proposed/desired algorithm may be directed to health event detection. The input parameters may be input features for a machine learning algorithm configured to predict a likelihood of a health event. HMS 22 may direct a remote computing system to execute logic for the proposed/desired algorithm of health event detection on the input samples/features and then, to return health event detection results.

As another example, the patient device may communicate, to HMS 22, a service request for access to and control of an approved algorithm for health event detection. Responsive to receiving the service request for a medical device of the patient, such as IMD 10, or another device, determining data indicative of one or more jurisdictional requirements corresponding to a patient location (e.g., previous or current patient location). The service request may include data identifying an algorithm for health event detection in patient data generated by IMD 10 or the other device. This may be a current algorithm in use for health event detection or a desired algorithm for possible future use. In accordance with the service request, HMS 22 accesses structured data including an approved usage scenario for the algorithm to determine whether the algorithm complies with the one or more jurisdictional requirements.

The approved usage scenario generally refers to a set of attributes describing a planned use of IMD 10 by its patient in compliance with the one or more jurisdictional requirements. By “attributes,” the present disclosure refers to information describing a jurisdiction, which may be a legal construct (e.g., state versus federal), physical location (e.g., patient location), a geographic area (e.g., European Union), or a designated authority by other jurisdictions (e.g., a regional body). Based on a determination that the algorithm complies with the one or more jurisdictional requirements, HMS 22 may automatically configure the patient data to be interoperable with the algorithm and when embodied in logic, its execution. HMS 22 may generate health event detection results based on an application of the algorithm to the configured patient data. Providing these results benefits the patient a great deal, for example, by ensuring the medical device operates in compliance with application laws.

A number of alternatives are possible from the present disclosure. Some alternatives may be expressed with different jurisdictional requirements. As described herein, an usage scenario may have its non-compliance overridden based on certain benefits outweighing the potential for patient harm. as datasets in the database system. As one alternative, instead of a private company's computing service technology, HMS 22 may be run by a government agency deliberating approval of any algorithm (e.g., a new or current algorithm) for use in patient assessments. As another alternative, a jurisdiction may not be tied to a locale or a specific physical location. Some jurisdictions may be defined by grouping smaller jurisdictions. An authority over validating medical devices in a region is one example.

As another example, the patient device may communicate, to HMS 22, a service request for automatic configuration of an approved algorithm. Based on a determination that an algorithm fails to comply with the one or more jurisdictional requirements, HMS 22 may select, from a plurality of algorithms, a compatible algorithm for performing an application to the patient data. HMS 22 may determine non-compliance of the algorithm based on a comparison, using a database system, between the algorithm and the one or more jurisdictional requirements. The database system arranges respective datasets for the plurality of algorithms. A dataset corresponding to the algorithm comprises approved usage scenario information for an application of the algorithm to the patient data. The comparison results may indicate a failure of the approved usage scenario information to satisfy the one or more jurisdictional requirements.

HMS 22 may identify the compatible algorithm from plurality of algorithms based on a comparison, using a database system, between the compatible algorithm and the one or more jurisdictional requirements. The database system arranges respective datasets for the plurality of algorithms. The dataset corresponding to the compatible algorithm comprises approved usage scenario information for an application to the patient data. The comparison results may indicate satisfaction by the approved usage scenario information of the one or more jurisdictional requirements.

Responsive to receiving a service request for a medical device, HMS 22 may determine data indicative of one or more jurisdictional requirements corresponding to a patient location. The service request may include data identifying an algorithm for health event detection in patient data generated by the medical device or another device. In accordance with the service request, HMS 22 may access structured data, in memory, storing an approved usage scenario for the algorithm to determine whether the algorithm complies with the one or more jurisdictional requirements. Based on a determination that the algorithm complies with the one or more jurisdictional requirements, HMS 22 may automatically configure the patient data to be interoperable with the algorithm. HMS 22 may generate the health event detection results based on an application of the algorithm to the configured patient data. By “configuring,” the present disclosure envisions modifying a format of a dataset, modifying which attributes to include in the dataset, and, possibly, modifying one or more attribute data values of the datasets.

In another example, HMS 22 may receive a service request and in response, access, from the memory, structured data of a usage scenario for an algorithm for health event detection in patient data generated by IMD 10. It should be noted that an association of the algorithm and the usage scenario in the structured data indicates that the usage scenario satisfies with one or more jurisdictional requirements. In response to the selection of the algorithm, HMS 22 may generate information operative to perform an automatic configuration of the patient data to be interoperable for logic implementing the algorithm (e.g., processor-executable instructions). HMS 22 may execute the logic for applying the selected algorithm to the automatically configured data and generate health event detection results.

In some examples, processing circuitry 50 transmits, via communication circuitry 60, the samples of patient data 82 (e.g., datasets of time-stamped patient activity such as electrical activity of a patient heart) for submission to computing system(s) 20 of FIG. 1. Patient data 82 may be partitioned (along a time axis) into samples of fixed or variable sample size. In one example, a sample of patient data 82 may include a certain length (measured in time) of recorded cardiac EGM data. Therefore, potential evidence may be included in a message indicating a possible occurrence of any health event. This message transmission may be in response to an initial indication of a cardiac episode; for at least that reason, the message transmission may request adjudication of the initial indication by a remote computing system for HMS. Transmission of the message may prompt adjudication by HMS 22. Communication circuitry 60 may include any suitable hardware, firmware, software, or any combination thereof for wirelessly communicating with another device, such as computing devices 12 and/or IoT devices.

The medical system and techniques of the present disclosure may package IMD 10 with external computing resources that results in enhancing IMD 10 in a number of ways. The medical system and techniques descried for FIG. 1 includes external computing devices with additional storage resources for patient data. The medical system and techniques described for FIG. 1 benefit from engaging a remote computing system to launch (remote) computing services instead of consuming local processing resources for new/updated software components. These capabilities may be new capabilities, firmware updates, support software and other components to improve the operations of IMD 10.

The following describes an example where HMS 22, when running validated algorithm(s) in its (server) device(s), may contribute to patient 4's medical care by supporting IMD 10's monitoring/detection functionality. HMS 22 may receive, via a communication protocol used for a memory uplink interrogation, readings/recordings of patient 4's physiological information from IMD 10 and after properly configuring the received information into compatible patient data suitable for a validated algorithm. IMD 10 may upload update(s) (e.g., also as a time sequence of samples) to the patient data stored by HMS 22. As directed by the validated algorithm, the server device of HMS may perform an analysis of (e.g., incoming) patient data samples and based on that analysis, detect the occurrence (if any) of the cardiac event.

HMS 22 may configure a (server) device to execute a validated algorithm on behalf of IMD 10, for example, by detecting an occurrence of a cardiac event or verifying an initial detection of a cardiac event by IMD 10. IMD 10 (e.g., periodically) uploads (e.g., as a time sequence) samples of previously (e.g., recently) captured physiological signals while capturing new physiological signals for patient 4 in performance of its monitoring/detection functionality. For each set of one or more uploaded samples, the server device runs the validated algorithm to evaluate the patient data in each sample for indicia of a cardiac event and, if available, to return any results data to IMD 10.

HMS 22 may communicate instructions directing IMD 10 to change its configuration to be compatible with characteristics of a validated algorithm. The device components of IMD 10 may have each of these requirements codified (e.g., in a software specification) as data elements that may be modified, added, and/or deleted to conform to the characteristics of the model. Logic within IMD 10 may be configured/re-configured to invoke the algorithm's functionality by providing, as input, the above-mentioned input features, storing, in memory, the above model components, and/or generating, to output, the above-mentioned output labels.

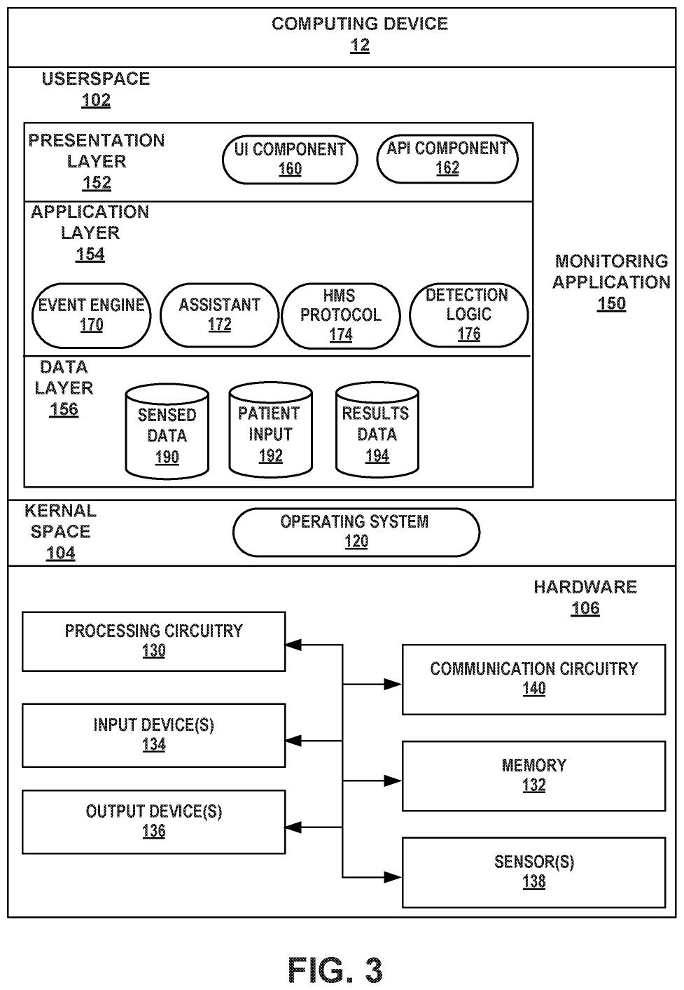

FIG. 3 is a block diagram illustrating an example configuration of a computing device 12 of patient 4, which may correspond to either (or both operating in coordination) of computing devices 12A, 12B, or 12C illustrated in FIG. 1. In some examples, computing device 12 takes the form of a smartphone, a laptop, a tablet computer, a personal digital assistant (PDA), a smartwatch or other wearable computing device. In some examples, computing device 12 may have a same or similar configuration to that of IMD 10 as illustrated in FIG. 2.

As shown in the example of FIG. 3, computing device 12 may be logically divided into user space 102, kernel space 104, and hardware 106. Hardware 106 may include one or more hardware components that provide an operating environment for components executing in user space 102 and kernel space 104. User space 102 and kernel space 104 may represent different sections or segmentations of memory, where kernel space 104 provides higher privileges to processes and threads than user space 102. For instance, kernel space 104 may include operating system 120, which operates with higher privileges than components executing in user space 102.

As shown in FIG. 3, hardware 106 includes processing circuitry 130, memory 132, one or more input devices 134, one or more output devices 136, one or more sensors 138, and communication circuitry 140. Although shown in FIG. 3 as a stand-alone device for purposes of example, computing device 12 may be any component or system that includes processing circuitry or other suitable computing environment for executing software instructions and, for example, need not necessarily include one or more elements shown in FIG. 3.

Processing circuitry 130 is configured to implement functionality and/or process instructions for execution within computing device 12. For example, processing circuitry 130 may be configured to receive and process instructions stored in memory 132 that provide functionality of components included in kernel space 104 and user space 102 to perform one or more operations in accordance with techniques of this disclosure. Examples of processing circuitry 130 may include, any one or more microprocessors, controllers, GPUs, TPUs, DSPs, ASICs, FPGAs, or equivalent discrete or integrated logic circuitry.

Memory 132 may be configured to store information within computing device 12, for processing during operation of computing device 12. Memory 132, in some examples, is described as a computer-readable storage medium. In some examples, memory 132 includes a temporary memory or a volatile memory. Examples of volatile memories include random access memories (RAM), dynamic random access memories (DRAM), static random access memories (SRAM), and other forms of volatile memories known in the art. Memory 132, in some examples, also includes one or more memories configured for long-term storage of information, e.g. including non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

One or more input devices 134 of computing device 12 may receive input, e.g., from patient 4 or another user. Examples of input are tactile, audio, kinetic, and optical input. Input devices 134 may include, as examples, a mouse, keyboard, voice responsive system, camera, buttons, control pad, microphone, presence-sensitive or touch-sensitive component (e.g., screen), or any other device for detecting input from a user or a machine.

One or more output devices 136 of computing device 12 may generate output, e.g., to patient 4 or another user. Examples of output are tactile, audio, and visual output. Output devices 136 of computing device 12 may include a presence-sensitive screen, sound card, video graphics adapter card, speaker, cathode ray tube (CRT) monitor, liquid crystal display (LCD), light emitting diodes (LEDs), or any type of device for generating tactile, audio, and/or visual output.

One or more sensors 138 of computing device 12 may sense physiological parameters or signals of patient 4. Sensor(s) 138 may include electrodes, accelerometers, an optical sensor, and other sensors, and sensing circuitry (e.g., including an ADC), similar to those described above with respect to IMD 10 and FIG. 2.

Communication circuitry 140 of computing device 12 may communicate with other devices by transmitting and receiving data. Communication circuitry 140 may include a network interface card, such as an Ethernet card, an optical transceiver, a radio frequency transceiver, or any other type of device that can send and receive information. For example, communication circuitry 140 may include a radio transceiver configured for communication according to standards or protocols, such as 3G, 4G, 5G, WiFi (e.g., 802.11 or 802.15 ZigBee), Bluetooth®, or Bluetooth® Low Energy (BLE).

As shown in FIG. 3, monitoring application 150 executes in user space 102 of computing device 12. Monitoring application 150 may be logically divided into presentation layer 152, application layer 154, and data layer 156. Presentation layer 152 may include a user interface (UI) component 160, which generates and renders user interfaces of health monitoring application 150. Presentation layer 152 may include an application programing interface (API) component 162, which instantiates an API with functionality for using software modules of application layer 154.

API component 162 may expose, via an interface, device logic for HMS to invoke in order to perform desired functionality. In one example, the computing system of the HMS may communicate a message directed to API component 162. The message may identify a function call, for example, for enforcing validation requirements for a current algorithm of detection logic 176.

Application layer 154 may include, but is not limited to, an event engine 170, an assistant 172, a monitoring service (HMS) protocol 174, and detection logic 176. Event engine 170 may be responsive to receipt of an indication of a current patient location by using HMS protocol 174 to submit a request for verification of detection logic 176 for the current patient location. This may occur when computing device 12 moves from a first geographic area (e.g., a country such as the United States) to a second geographic area (e.g., a non-US country). In general, detection logic 176 may be characterized as programming configured to determine whether there is a sufficient likelihood that patient 4 is experiencing a health event for a time period encompassing one or more samples. Lastly, assistant 172 may represent software/hardware component(s) for generating a user interface for a device user such as a patient and/or a clinician to avail.

Event engine 170 may control performance of device functionality including any operation by detection logic 176 in response to (e.g., one or more samples of) sensed data 190, for example, if detection logic 176 implements an algorithm whose programming involves an application of a model (e.g., a machine learning algorithm). Event engine 170 may be configured to support detection logic 176 by conforming its programming to accept input for the model. As described herein, detection logic 176 may be configured correctly to receive input for a current model but a same configuration may be incompatible for receiving input for a different model. This may be because the current model and the different model process, as input, different configurations of sensed data 190. Event engine 170 may update the programming of detection logic 176 to implement for detection logic 176 a modified input interface that matches the configuration of the different model.

Detection logic 176 may be programmed according to an algorithm for maintaining, in memory, a structure representing a machine learning model; when prompted by input from another application, detection logic 176 may be configured to apply the model using the structure in a series of logical steps to one or more samples of input data and then, to predict a health event. The model may be configured to determine whether a sample provides significant evidence of a health event. Detection logic 176 may output data indicative of the prediction rendered by the algorithm. The input data and/or the output of the algorithm/model may each have a certain configuration (e.g., format) that differs from each other and other model data. For at least this reason, the present disclosure provides event engine 170 to alter the configuration of the input data, the output data, or both to conform to a new algorithm.

To illustrate by way of example, event engine 170 may replace input features in memory for the current model with new input features of the different (second) model. The present disclosure envisions other operations that event engine 170 may perform to adapt application layer 154 (e.g., any component thereof including detection logic 176) to an algorithm for the different model. The HMS described in the present disclosure may provide information to effectuate the automatic configuration. This information may include instructions (e.g., processor-executable instructions) that, when executed, loads suitable logic to alter the programming of device logic (e.g., detection logic) in application layer 154.

In some examples, event engine 170 receives from HMS automatic configuration information, which outlines steps for verifying the current algorithm implemented in detection logic 176 and its programming. The HMS automatic configuration information may include optional steps for replacing the programming of the detection logic with new program code. As described herein, a medical professional or the device user may select a different algorithm for use in a medical device such as IMD. Clinician 8 may desire using the different algorithm as part of a clinical trial where patient 4 is a clinic patient as well as the device user. The desired algorithm may not have been approved by a relevant authority of the current patient location. Assuming the clinical trial has been validated by the relevant authority, clinician 8 may override the lack of approval and use the desired algorithm to replace the current algorithm implemented by detection logic 176. Via HMS protocol 174, messages may be communicated to effectuate the verification and override. Event engine 170 may retrieve (e.g., download) program code of the desired algorithm to be loaded into memory space for detection logic 176.

In some examples, event engine 170 executes detection logic 176 to analyze sensed data 190 and, in some examples, patient input 192, and then, generate results data 194 indicative of an initial detection of a cardiac episode or a confirmation of the initial detection as true positive. Results data 194 may also store information for each rejection of the initial detection.

Event engine 170 may be responsive to receipt of a transmission from IMD 10 including physiological data to store in sensed data 190. Event engine 170 may include device logic configured to generate, from the sensed data 190, samples to be feed, as input, into detection logic 176. The machine learning algorithm implemented by detection logic 176 may, based on one or more input samples, determine whether a health event occurred, is occurring, or will occur. The device logic of event engine 170 may store a determination by detection logic 176 in results data 194.

Event engine 170 may be responsive to receipt of a transmission from IMD 10 indicating that IMD 10 detected a health event, e.g., a cardiac episode such as an arrythmia. Event engine 170 may activate detection logic 176 and/or HMS to perform an adjudication of the detection by IMD 10.

Sensed data 190 may include data received from IMD 10 as part of a transmission, additional data transmitted from IMD 10, e.g., in “real-time,” and physiological and other data related to the condition of patient 4 collected by computing device(s) 12 and/or other devices. As examples, sensed data 190 from computing device(s) 12 may include one or more of: activity levels, walking/running distance, resting energy, active energy, exercise minutes, quantifications of standing, body mass, body mass index, heart rate, low, high, and/or irregular heart rate events, heart rate variability, walking heart rate, heart beat series, digitized ECG, blood oxygen saturation, blood pressure (systolic and/or diastolic), respiratory rate, maximum volume of oxygen, blood glucose, peripheral perfusion, and sleep patterns.

Patient input 192 may include appropriate information in response to patient queries, for example, regarding symptom(s). To illustrate, Patient input 192 may include responses to queries posed by health monitoring application 150 regarding a current condition of patient 4, input by patient 4 or another user, such as clinician 8. The queries and responses may occur responsive to entering a geofenced area and receiving a message from a device of HMS 22, e.g., as part long-term monitoring of the health of patient 4. User recorded health data may include one or more of: exercise and activity data, sleep data, symptom data, medical history data, quality of life data, nutrition data, medication taking or compliance data, allergy data, demographic data, weight, and height. EHR data 194 may include any of the information regarding the historical condition, symptoms, or treatments of patient 4 described above. EHR data may relate to history of cardiac arrest, tachyarrhythmia, myocardial infarction, stroke, seizure, chronic obstructive pulmonary disease (COPD), renal dysfunction, or hypertension, history of procedures, such as ablation or cardioversion, and healthcare utilization. EHR data 194 may also include demographic and other information of patient 4, such as age, gender, height, weight, and BMI.

Patient 4, who is a user of a medical device, such as IMD 10 of FIG. 1, may prompt IMD 10 to generate sensed data 190 from various signals. In further response to a prompt from patient 4 and/or clinician 8, monitoring application 150 may configure computing device 12 for synchronizing sensed data 190 (including device interrogation data) and/or results data 194 with an external storage location such as database 24.

An assistant 172 may provide a conversational interface for patient 4 and/or clinician 8 to exchange information with computing device 12. Event assistant 176 may query the user regarding the condition of patient 4. Responses from the user may be included as patient input 192. Event assistant 172 may use natural language processing and context data to interpret utterances by the user. In some examples, in addition to receiving responses to queries posed by the assistant, assistant 172 may be configured to respond to queries posed by the user. In some examples, event assistant 172 may provide directions to and respond to queries regarding treatment of patient 4 from patient 4 or clinician 8.

Event engine 170 may employ a location service (not illustrated) to determine the location of IMD 10 and/or computing device 12 and, thereby, the presumed location of patient 4. Location service may use global position system (GPS) data, multilateration, and/or any other known techniques for locating computing devices.

In general, HMS protocol 174 controls the exchange of messages with a (local or remote) monitoring service running in a computing system. The monitoring service (HMS) may prompt computing device 12, via monitoring application 150, to perform one or more operations, thereby modifying or replacing detection logic 176 with logic for an approved algorithm that is different from the current algorithm.

FIG. 4 is a block diagram illustrating an operating perspective of HMS 22. HMS 22 may be implemented in a computing system 20, which may include hardware components such as those of computing device 12, embodied in one or more physical devices. FIG. 4 provides an operating perspective of HMS 22 when hosted as a cloud-based platform. In the example of FIG. 4, components of HMS 22 are arranged according to multiple logical layers that implement the techniques of this disclosure. Each layer may be implemented by one or more modules comprised of hardware, software, or a combination of hardware and software.

Computing devices, such as computing devices 12, may operate as clients that communicate with HMS 22 via interface layer 200. The computing devices typically execute client software applications, such as desktop application, mobile application, and web applications. Interface layer 200 represents a set of application programming interfaces (API) or protocol interfaces presented and supported by HMS 22 for the client software applications. Interface layer 200 may be implemented with one or more web servers.

As shown in FIG. 4, HMS 22 also includes an application layer 202 that represents a collection of services 210 for implementing the functionality ascribed to HMS herein. Application layer 202 receives information from client applications (e.g., a verification request for detection logic employed by a medical device such as IMD 10, an alert of a health event from computing device 12A, and/or the like) and further processes the information according to one or more of the services 210 to respond to the information. Application layer 202 may be implemented as one or more discrete software services 210 executing on one or more application servers, e.g., physical or virtual machines. That is, the application servers provide runtime environments for execution of services 210. In some examples, the functionality interface layer 200 as described above and the functionality of application layer 202 may be implemented at the same server. Services 210 may communicate via a logical service bus 212. Service bus 212 generally represents a logical interconnections or set of interfaces that allows different services 210 to send messages to other services, such as by a publish/subscription communication model.

Data layer 204 of HMS 22 provides persistence for information in PPEMS using one or more data repositories 220. A data repository 220, generally, may be any data structure or software that stores and/or manages data. Examples of data repositories 220 include but are not limited to relational databases, multi-dimensional databases, maps, and hash tables, to name only a few examples.

As shown in FIG. 4, each of services 230-238 is implemented in a modular form within HMS 22. Although shown as separate modules for each service, in some examples the functionality of two or more services may be combined into a single module or component. Each of services 230-238 may be implemented in software, hardware, or a combination of hardware and software. Moreover, services 230-238 may be implemented as standalone devices, separate virtual machines or containers, processes, threads or software instructions generally for execution on one or more physical processors.

Input/output processor 230 may be responsive to receipt of a transmission (e.g., an alert transmission) from computing device(s) 12 indicating that the medical device (e.g., IMD 10) detected a health event of patient and, in some examples, that the transmitting (intermediary) patient device confirmed the detection as a true positive upon adjudication. Input/output processor 230 may initiate further adjudication of the detection health event. Input/output processor 230 may initiate performance of any of the operations in response to detection of the health event to HMS 22, such as communicating with patient 4, clinician 8, and care providers within a facility, and, in some cases, analyzing data to confirm or reject the detection of the health event by IMD 10.

Input/output processor 230 may coordinate an automatic configuration of IMD 10 for the device user's intended use of a new algorithm, for example, given that a current algorithm implemented by device logic is to be replaced. Input/output processor 230 may provide IMD 10 and/or computing device 12A with logic implementing the new algorithm that is loaded into device memory for updating the programming for the current algorithm.

HMS 22 may provide information for the automatic configuration. IMD 10 may submit a message to input/output processor 230 identifying the new algorithm and requesting the automatic configuration. As an option, input/output processor 230 may receive the above message further requesting verification of the new algorithm as being compliant with a local jurisdiction's rules and to conclude the verification prior to communication of any automatic configuration information. If the new algorithm has not yet been approved, input/output processor 230 may return a reply message describing the lack of approval, unless overruled by IMD 10 and/or computing device 12. If the new algorithm is to be used in a clinical setting, input/output processor 230 may accept the new algorithm in accordance with the validation requirements and override the previous non-approval.

Record management service 238 may store a variety of patient data including input features for machine learning models and samples of raw or processed physiological data of the patient. Record management service 238 may package the patient data into each (database) record, in some cases with additional information as described herein, including any messages exchanged on behalf of the patient, such as messages for transmission to clinician 8 and/or care providers.

In examples in which HMS 22 performs an analysis to confirm or override the detection of the health event by IMD 10, input/output processor 230 may apply one or more validation rules 256 to a model that the new algorithm instruments for the purpose of computing a likelihood of any given health event, such as a cardiac episode. Some examples of validation rules 256 include geographic (e.g., country) restrictions.

Code library 252 generally includes identifying logical units (e.g., programs) for a number of algorithms that can be implemented in a medical device. Input/output processor 230 may retrieve a logical unit for the new algorithm and return the logical unit in a reply message transmitted to IMD 10. The logical unit may include packaged instructions that are processor-executable; when communicated to IMD 10, client application 72 may load these instructions into memory and update IMD 10's programming (e.g., detection logic). Additional instructions may be packeted into the logical unit such that the execution of these instructions causes changes to an input interface to the detection logic. These changes allow the detection logic to accept, as input, compatible feature vectors for the new algorithm instead of incompatible ones for the replaced algorithm.

Code library 252 may include one or more models, algorithms, decision trees, and/or thresholds, which may be developed by model configuration service 234 based on machine learning. Example machine learning techniques that may be employed to generate rules can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, the Apriori algorithm, K-Means Clustering, k-Nearest Neighbor (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

In some examples, model configuration 234 may be configured to modify components (e.g., parameters, rules, and/or the like) of the given model based on feedback data 254 that indicates whether the detections and confirmations of health events by IMD 10, computing device 12, and/or HMS 22 were accurate. Feedback data 254 may correspond to adjudications of initial detections of health events by IMD 10 and/or computing device 12. Feedback data 254 may further refine the data corresponding to the above adjudications for their corresponding algorithms/models implemented in code library 252. Feedback data 254 may be used to improve upon performance of any algorithm and/or their specific model(s) by way of training. Training may involve modifying the programming in code library 252 of any algorithm/model. In some examples, true and false detections (as indicated by event feedback data 254) and confirmations for supervised machine learning may be used to further train models.

HMS 22 may direct a client application to automate the above verification, for instance, when patient 4 is in a clinical setting for area 6 and the usage scenario is pre-defined. For instance, computing device 12A may include a transmitter that can interrogate IMD 10 and by way of that transmitter, have interrogated device data transmitted to HMS 22 via a connection that is either wireless or wired such that the data can be reviewed remotely by physicians, device clinic personnel, and device manufacturer personnel.

HMS 22 may direct computing system 20A (or computing system 20B) to communicate with a hardware/software component of computing device(s) 12 and retrieve any additional patient data. HMS 22 may utilize the additional patient data when performing the new algorithm. HMS 22 may also retrieve data regarding patient 4 from one or more sources of electronic health records (EHR) via network. EHR may include data regarding historical (e.g., baseline) physiological parameter values, previous health events and treatments, disease states, comorbidities, demographics, height, weight, and body mass index (BMI), as examples, of patients including patient 4. HMS 22 may use data from EHR to configure algorithms implemented by IMD 10 and/or computing devices 12 to detect health events for patient 4. In some examples, HMS 22 provides data from EHR to computing device(s) 12 and/or IMD 10 for storage therein and use as part of their algorithms for detecting health events.

As illustrated in the example of FIG. 4, services 210 may also include an assistant configuration service 236 for configuring and interacting with assistant 172 of FIG. 3. The patient may enter information via an interface for submission to HMS 22 over a network connection. This submission may cause HMS 22 to initiate an action that involves a remote computing system. In some examples, HMS 22 may desire cardiac activity data from the patient's device(s) and record that data as ECG/EGM data 250.

In some examples, HMS 22 may operate within a geographic area designated for providing medical care to the patient (e.g., a geofenced geographic area). The medical systems and techniques may implement geofencing technology to enable monitoring service 250 to recognize certain events, such as when the patient visits, for example, the clinical setting. A geofence can be configured for the clinic using geographic coordinates. The local monitoring service may be configured using a number of technologies and as such, may be configured with a GPS module operative to receive coordinates of the patient at their current location and/or coordinates of the clinic. Based on a comparison between both sets of coordinates, HMS 22 may determine when the patient is currently located in the clinic and then, responsive to the determination, initiate an automatic verification of a current algorithm or a proposed new algorithm for replacing the current algorithm.

HMS 22 may be configured to run, over the network, on computing system(s) 20, for example, to enforce compliance on patient devices, such as IMD 10 and/or computing device 12A. HMS 22 may store information indicating applicable laws, regulations, and other rules for each locality (e.g., country). As described herein, a country or jurisdiction can create an agency with the power to approve/disapprove IMD 10 for general public use prior to their commercial sale; that agency's approval/disapproval power extends to specific algorithms that IMD 10 may run as part of its programming or that HMS 22 may run as part of its cloud service. By defining each algorithm using a number of parameters, HMS 22 may change the algorithm and/or the patient data on which the algorithm is applied by modifying stored parameter values, causing conformity between the patient data and logic for evaluating the patient data.

In one example, HMS 22 may use containerization to enable the evaluation operation by availing benefits of using a secure and protected execution environment. HMS 22 may translate a corresponding machine learning algorithm/model into logic or code and then, execute the logic or code via one or more processors. HMS 22 may perform the evaluation operation on a set of input samples and for each sample subset of one or more samples, generates output indicative of a prediction (e.g., a probability such as a likelihood give the input samples). While most of the samples are not incidents of cardiac episodes (e.g., arrythmias), one or more samples may provide sufficient evidence of actual occurrences of such episodes. Accordingly, HMS 22 may warn patient 4 and/or his/her clinician 8 by generating an alarm and/or communicating a message to notify patient 4.

In some examples, an external device or system (e.g., computing device(s) 12 or computing system(s) 20 of FIG. 1) may perform the evaluation operation as described herein. This operation may be an adjudication following an initial detection; in effect, the external device or system determines whether the initial detection was a false positive/true positive. Similar to HMS 22, IMD 10 or patient computing device 12 perform the evaluation operation on a set of input samples and for each sample subset of one or more samples, generates output indicative of a prediction (e.g., a probability such as a likelihood give the input samples). While most of the samples are not incidents of cardiac episodes (e.g., arrythmias), one or more samples may provide sufficient evidence of actual occurrences of such episodes.

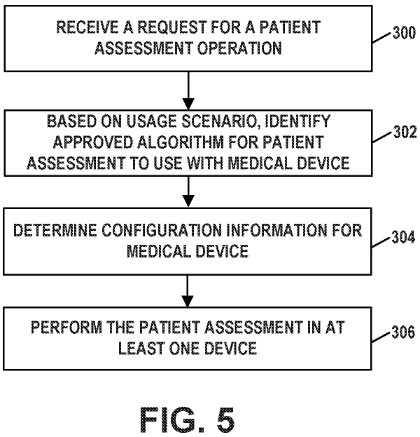

FIG. 5 is a flow diagram illustrating an example operation by a computing device of a medical system that operates in accordance with one or more techniques of the present disclosure. The example operation depicted in FIG. 5 is described with respect to a computing device 12 depicted in FIGS. 1 and 3, but may be described with respect to any one or more devices, e.g., computing devices 12, computing systems 20, IMD 10, or monitoring service (MS) 22 of FIGS. 1-4 that may implement a monitoring service and/or a client or monitoring application for the monitoring service. The techniques described herein may be performed by processing circuitry of any one or more of these devices.

A monitoring service may be configured as a computing service (e.g., a cloud service) that is accessible over a network from any geographic area using a number of communication protocols as described herein such as the Internet. The monitoring service may combine a local area network and server with a network service running on a network-accessible resource and built using network functions that are compatible with a number of protocols. The network-accessible resource at the external location may be used to store patient data and facilitate the patient's medical care. Similar to the local area network, the remote computing system may initiate a main process of the monitoring service to perform a number of operations including medical device verification and automatic configuration for use of an approved algorithm for native device functionality.

According to the illustrated example of FIG. 5, processing circuitry of system 2 (e.g., processing circuitry of a similar computing device to computing device(s) 12 and also a part of computing system(s) 20 of HMS 22) may configure a computing service to run over a network on a computing system as a monitoring service for enforcing statutory and regulatory compliance on medical devices. The same computing system may provide a different (second) computing service for health event monitoring based on a patient's physiological data (e.g., sensed data 190) generated by sensing circuitry (e.g., sensing circuitry 52 of IMD 10) and patient input (e.g., patient input 192). According to the illustrated example of FIG. 5, the processing circuitry verifies that an algorithm used by IMD 10 to detect an arrhythmia or a cardiac arrest complies with applicable rules of a geography within which IMD 10 is being used (e.g., a current or future patient location), thereby ensuring only approved medical care is being given to a population within the geography.

The above monitoring service, as an option, may be configured to detect changes in the health of patients based upon patient data. For example, by way of adjudication, the computing system running the monitoring service may perform an assessment of any patient with as pacemakers, defibrillators, monitors, and other examples of implantable medical device(s) (e.g., IMD 10 of FIG. 1).

The patient device may be a mobile device configured to communicate (e.g., interrogate the medical device of the patient and on behalf of that medical device, exchange messages with the monitoring service and/or the second computing service. As an alternative, the medical device may be configured with hardware/software for directly exchanging messages with the monitoring service.

A patient may accept health monitoring to some degree beyond their (personal) device, such as from an external device or a remote computing system running over a network; however, regardless of which device actually executes instructions of an algorithm configured to detect occurrences of health events, the algorithm is required to be an approved algorithm for medical devices. Geographic areas such as countries each designate an agency to be an authority for the medical devices.

To commence the example operation of FIG. 5, the processing circuitry receives an indication of a message for the monitoring service (300). The message may be a request from a medical device for an automatic configuration. The monitoring service may defines the configuration of medical device to include any algorithm behind device functionality. In some examples, the medical device provides a patient with certain health event detection capabilities. In some examples, the medical device provides functionality in which one or more operations are based on machine learning, such as training/testing a model or evaluating samples by applying a model. The message may be arranged into packetized data in accordance with any communication technology (e.g., protocol).

Based on information describing a usage scenario intended for the medical device while using the particular algorithm, the processing circuitry may identify an approved algorithm for use in the medical device for a patient assessment as part of health event monitoring/detection (302). In the example of FIG. 5, the processing circuitry determines that the above message is requesting an automatic configuration where the medical device detects a variety of cardiac events using a particular algorithm. The particular algorithm may be a machine learning algorithm that includes a number of operations of which one may be a model evaluation of an input sample for predicting a probability of a particular cardiac event occurring at a corresponding time period. the monitoring service determines whether such an intended use complies with applicable rules governing a local geography. These rules vary in terms of government oversight and authority.

In general, the usage scenario describes the intended use for the particular algorithm while programmed into device logic (e.g., as detection logic). The usage scenario may identify a geographic area, such as a country, where the patient intends to use the medical device with the particular algorithm. The usage scenario may further identify a setting intended for the patient's use. This setting may be a clinical setting where the patient is part of a clinical trial or a general setting where the patient is not part of any clinical trial. The trial may further include being monitored in a facility. The usage scenario may specify which model is being implemented. For each of the above elements of the usage scenario, there may one or more applicable rules.

To illustrate by way of example, the particular algorithm may be approved for the medical device in one country but a second country may not permit any use of the medical device and/or the particular algorithm. A third country may decide to approve of the particular algorithm but only with a specific machine learning model. A fourth country may restrict the particular algorithm to a clinical setting where the patient is being monitored. Moving between countries may require an automatic configuration using an approved algorithm. The automatic configuration may be automated upon the medical device and/or the patient entering the new country. Each re-configuration, the processing circuitry modifying the programming within the device logic to confirm device components (e.g., input/output memory buffers) to the input interface(s) and output interface(s) of the logic implementing the particular algorithm.

In response to determining that the particular algorithm is an approved algorithm for the medical device, the processing circuitry may determine configuration information for the medical device to implement prior to the implementing any approved algorithm including the particular algorithm (304). In general, the configuration information effectuates the automatic configuration of the medical device for facilitating incorporation of logic implementing the particular algorithm. As described herein, this may include modifying/creating a structure in memory to conform device logic with an input interface of the logic implementing the particular algorithm.

The patient device(s) may initiate transmissions to provide the remote computing systems with datasets of the most recent patient data, including sensed patient data generated by the patient's medical device, in performance of the device interrogation by the monitoring service and/or the second computing service. The processing circuitry may perform the patient assessment by executing the health even detection logic within the medical device, the patient device(s), and/or the remote computing system(s) (306).

The order and flow of the operation illustrated in FIG. 5 are examples. In other examples according to this disclosure, more or fewer thresholds may be considered. Further, in some examples, processing circuitry may perform or not perform the methods of FIG. 5, or any of the techniques described herein, as directed by a user, e.g., via external device 12 or computing devices 100. For example, a patient, clinician, or other user may turn on or off functionality for identifying changes in patient health (e.g., using Wi-Fi or cellular services) or locally (e.g., using an application provided on a patient's cellular phone or using a medical device programmer).

FIG. 6 is a diagram illustrating an example process in which profile 400 is used in first operation 500 and second operation 600. First operation 500 may be directed towards generating profile 400 for use in a verifying a given algorithm as valid or approved for patient use under a local jurisdiction. Second operation 600 may be directed towards referencing profile 400 for use in configuring datasets of parameter values and other information corresponding to a patient into compatible patient data for a given algorithm.

In one example, first operation 500 may generate profile 400 in response to a service request from a computing device for patient 4 or clinician of FIG. 1, unless an adequate copy of profile 400 is available to handle the service request. As described herein, profile 400 may include information identifying applicable validation requirements for the local jurisdiction. One validation requirement may prescribe a rule that a given algorithm must be compatible with patient data as stored in memory. The prescribed rule may further require that logic for the given algorithm be configured (e.g., programmed with appropriate settings) such that processing circuitry executing the logic is capable of accessing, via a schema, specific parameters of a dataset in the patient data. The schema described herein refers to a structure for organizing the patient data stored in the memory,

The present disclosure describes herein a “schema” or a “schematic representation” as generally referring to a structure in which parameters and other structural components are combined (e.g., forming an address space in memory) for efficient storage and retrieval, for example, by a computing service. A jurisdiction governing a location may prescribe rules (e.g., constraints) for analyzing patient data (e.g., as a machine learning model). The location of the jurisdiction may be any geographic location such as a current or previous patient location, a medical device location, or a location of a number of computing device for a monitoring service, an example computing service, as such HMS 22 of FIG. 1. Underlying the monitoring service, the number of computing devices, operating as servers, and networking infrastructure cooperate to expose, over network 16, an interface for receiving message communications from a user device of a patient or the patient's clinician. The servers of the monitoring service may be configured to process the message communications as service requests.

HMS 22 may be operative to handle requests (e.g., from a clinician or a manufacturer of IMD 10) for an automatic configuration of at least one of information/data, a medical device, and/or an executable element for an algorithm. In one example of the automatic configuration of data, an appropriate configuration for datasets of patient data generated by IMD 10 may be sets of input arguments for serial function calls on an interface (e.g., an Application Programming Interface (API) for the executable element for the algorithm. In an example where IMD 10 includes device logic compatible with a machine learning model, the input arguments may be input features for the model, and therefore, HMS 22 may convert the patient data into input feature vectors. In one example of the automatic configuration of the executable element, an appropriate configuration for logic for executing the algorithm may be an updated interface or input contract or compatible parameters (e.g., model parameters). HMS 22 may alter program code to effectuate the appropriate configuration. In one example of the automatic configuration of the medical device, an appropriate configuration for IMD 10 (e.g., where device logic is compatible with a machine learning model), HMS 22 may instruct IMD 10 to update its programming, for example, by modifying device settings, parameters, and other controls. The device logic of IMD 10 may change the machine learning model by changing an object definition of the particular model being implemented. In one example, IMD 10 may implement a set of programmable parameters to configure the device logic to be interoperable with logic for implementing the model. IMD 10 may use the device logic to provide a logical layer through which input features, model components, or output labels are added, removed, or modified in response to HMS 22 submitting a new set of programmable parameters for defining a new model for IMD 10 to implement. In turn, the logical layer may translate the submitted parameter data into instructions for changing the current programmable parameters of the device logic. Implementing the new model can be accomplished in a number of other ways.

A device may have an approved algorithm for application to the datasets of patient data and execute logic (e.g., in a software program) to run that approved algorithm. In one example, first operation 500 may configure (e.g., modify or rearrange) contents of the datasets into (re)configured datasets that are compatible with an interface to the approved algorithm (as an input feed. The interface may be run as an executed software program having code points (e.g., entry points in specific memory locations) for receiving input data (e.g., input arguments in an invocation of a specific function call) for prompting an application of the approved algorithm to the input data. Message communications as described herein may include the input data for the function call.

Structure 410, a data schema or format for implementing profile(s) 400 in storage 420, may define an example arrangement of a number of attributes of which at least one attribute identifies an approved algorithm for a specific device type. Storage 420 includes computer memory for storing a number of various profiles that are each generated according to the structure 410. A number of different profile embodiments are envisioned herein by the present disclosure for enabling a search/identification and then, possible configuration of one or more approved algorithms for the specific device type.

The following description refers to profile 400 as device profile 400 in which attribution information indicates a number of approved algorithms (e.g., algorithm versions) for the specific device type of the medical device. In accordance with structure 410, an organization of the attribution information in each device profile 400 facilitates the above-mentioned search/identification and/or configuration. As illustrated, the attribution information of profile 400 may further indicate one or more requirements for the medical device to satisfy before using an approved algorithm. For each requirement, the attribution information of profile 400 may also indicate an appropriate and satisfactory configuration of the medical device to generate parameterized patient data.

As illustrated in FIG. 6 by respective flow diagrams for first operation 500 and second operation 600 of the example process, a medical system creates device profile 400 to enable an appropriate configuration of patient data for an approved algorithm and, possibly, to effectuate an efficient and low-latency application of the algorithm on the configured patient data to detect an occurrence of a health event. Device profile 400 may store, in configuration information, a dataset of characteristics of the algorithm (e.g., requirements for input/output of a machine learning model) and mapping between the dataset and specific device configuration details (e.g., device settings, parameters, modes, and/or the like). First operation 500 generates device profile 400 to include such configuration details as an appropriate configuration for (e.g., patient data generated by) devices (e.g., medical devices) of that specific device type. Second operation 600 references device profile 400 to pre-process the patient data prior to applying the algorithm to the pre-processed patient data generated by one or more devices matching the specific device type. Pre-processing may be performed to correct an incompatibility between the algorithm and the patient data.

To illustrate, a medical device such as IMD 10 may generate patient data using raw device measurements and/or sensed physiological input, however, in some examples, such data may not be compatible with the characteristics of the algorithm, inhibiting an application of the algorithm to the raw device measurements and/or sensed physiological input; therefore, by pre-processing the patient data for the approved algorithm according to parameters and other appropriate configuration details defined in device profile 400, the medical system may conform the patient data to be compatible to the algorithm. The medical system may parametrize the raw device measurements and/or sensed physiological input such resulting algorithm compatible datasets of device parameter data may be interoperable with logic for executing the algorithm. The medical system may use one or more algorithm compatible datasets to form input (e.g., arguments including input model features) for function calls (e.g., evaluations of a machine learning model) with the executed logic. As an option, the medical system may feed, as input, the pre-processed patient data into an interface of logic for executing the algorithm.

Health monitoring service (HMS) 22 of FIGS. 1-4 represents an example medical system, and the example operations depicted in FIG. 6 are described with respect to a computing device of computing system(s) 20. One or both of first operation 500 and second operation 600 as described herein may be performed by processing circuitry of the computing device.

To commence first operation 500 of FIG. 6, HMS 22 receives an indication of a message in which a user of a patient device enters an approved algorithm for a country or a clinic corresponding to that patient device or another computing device (510). The patient device may be a specific type of medical device (e.g., IMD 10 of FIG. 1) or a computing device programmed to operate as a specific type of medical device, such as a patient device (e.g., a mobile device such as computing device 12 of FIG. 1) or a server device as part of a computing system (e.g., computing system 20 of FIG. 1) for running a computing service over a network connection.

HMS 22 retrieves algorithms available for an implanted device (520). As further described for FIG. 6, device profile(s) 400 may store information enabling HMS 22 to ensure approved medical devices operate in compliance with the one or more requirements (e.g., jurisdictional requirements). An algorithm may be approved for the implanted device in public use in the United States while another algorithm may not be approved for public use but may be approved for the same implanted device in a clinical setting.

HMS 22 creates an implanted device specific algorithm configuration based on country and clinic configurations (530). As described herein, structure 410 determines an organization of the attribution information in each device profile 400 to facilitate the above-mentioned search and identification. Structure 410 may further define attributes for an appropriate configuration of any device type, including the implanted device specific algorithm configuration of FIG. 6.

Consider an example where HMS 22 of medical system 2 of FIG. 1 receives, from a medical device of a specific device type, a service request regarding a planned usage scenario of a particular algorithm (e.g., with the device's patient). Given certain search elements selected from the usage scenario—including a country and/or a setting (e.g., a clinical or public setting)—HMS 22 may identify one or more approved algorithms. In some examples, HMS 22 may verify the usage scenario as valid and communicate approval of the service request or, vice versa, may determine that the usage scenario renders the medical device invalid and returns a disapproval/denial of the service request. If the particular algorithm matches one of the one or more approved algorithms, the service may return an appropriate device configuration (if needed) for the medical device to be compatible with that approved algorithm. In turn, the medical device uses attributes of the appropriate device configuration to conform a current device configuration, for example, by reprogramming device logic to be interoperable with logic for executing the corresponding approved algorithm. If storage 420 includes a number of approved versions of the particular algorithm, HMS 22 may return information identifying each approved version.

To commence second operation 600 of FIG. 6, HMS 22 determines that patient data needs to be processed by an ML algorithm (610). As described herein, medical device may employ algorithms based in machine learning concepts and may train and/or evaluate a machine learning model to predict health events.

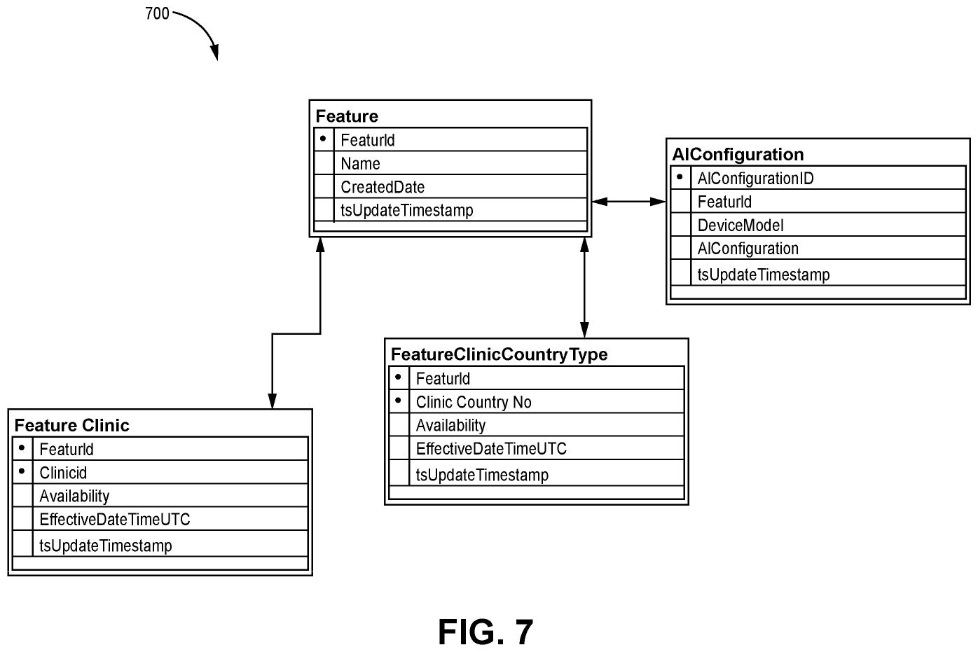

MS retrieves an algorithm configuration (620) from a corresponding profile 400 in accordance with structure 410, an example of which is further illustrated in FIG. 7. The corresponding profile 400 may be configured as a device profile or, as an alternative, an algorithm profile that is agnostic to device type. HMS 22 pre-processes the patient data to confirm to the interface/input contract for the configured ML algorithm (630) and then, processes the patient data with the configured ML algorithm. Storage 420 includes configuration information for transforming the patient data into input features for the above model. HMS 22 may include a code repository with modules for different algorithms from which a module may have health event detection logic for the configured ML algorithm. The module may receive, as input, the input features, execute the detection logic to evaluate the above model, and generate, as output, a label predicting a health event. HMS 22 may pre-process the patient data to generate datasets of the input features for submission, via the interface, to the module.

In contrast to operation 600, the configuration information stored in storage 420 corresponds to configuring device logic in the patient device such as the medical device of operation 500. In the patient device, the patient device may update the device logic to implement the health event detection logic for the configured ML algorithm. There are a number of other techniques for configuring the patient device to conform to the configured ML algorithm. Similar to the computing device of HMS 22, the patient device may configure the device logic to pre-process the patient data into datasets of the input features for submission, via an interface, to the health event detection logic (or simply “detection logic”). As an alternative, HMS 22 may provide the pre-processed patient data to the patient device for the submission, via the interface, of the input features to the detection logic.

FIG. 7 is a diagram of database schema 700 for profile 400 of FIG. 6, in accordance with one or more examples of the present disclosure. Database schema 700 defines a structure to format various information of an example of a profile as described herein. For any given profile, the structure may define different sets of requirements for using an algorithm in a specific device type or for computing devices in general. Each set of requirements may correspond to a specific usage scenario and/or include an appropriate configuration for a device in that specific usage scenario.

As illustrated, database schema 700 defines independent sets of requirements for two usage scenarios: A generic clinic (illustrated in FIG. 7 as table “FeatureClinic”) and a clinic of a certain country (illustrated in FIG. 7 as table “FeatureClinicCountryType”). Database schema 700 defines an appropriate configuration for any patient device that satisfies the other requirements for one of above two usage scenarios (illustrated in FIG. 7 as table “AIConfiguration”).

A medical system may define attributes of database schema 700 and populate a number of profiles for different algorithms and, possibly, specific medical devices on which an algorithm is executed. For each algorithm/device profile, the medical system store attribution data based on requirements (e.g., validation requirements) prescribed by a relevant authority of a local jurisdiction.

In the local jurisdiction, a regulatory body in charge of promulgating the requirements for the above two usage scenarios may be a government agency. That regulatory body may be the relevant authority charged with validating various types of medical devices and their algorithms used for patient care. To explain by way of example, if a medical device (or, as an alternative, the monitoring service itself) executes specific (detection) logic for performing an assessment of relevant patient data (i.e., a patient assessment) and detecting any occurrences/evidence of cardiac episodes in that patient data, the monitoring service, prior to the medical device performing the patient assessment, must ensure that the detection logic complies with the applicable laws of the patient's country. Given that the monitoring service operates over a network (i.e., as a network service), the service may direct a patient's cardiac monitoring device to perform the patient assessment after making sure the detection logic has been validated for use in medical devices by all local government agencies. The monitoring service, similarly, may verify the algorithm when the patient assessment is performed over the network.

The detection logic may be defined in terms of an algorithm enabling cardiac episode detection by implementing various functionality, and the above profiles may be used to model output provided by the detection logic. Leveraging these profiles, the monitoring service can evaluate performance of any algorithm. One example is a machine learning evaluation in which predicting a specific variable involves an application of a machine learning model (e.g., a neural network) on a sample dataset. The specific variable as described herein may refer to a likelihood or a Boolean indicative of an occurrence of a cardiac episode or another parameters indicative of patient health. The sample dataset refers to N-dimensional data partitioned into a number of samples of which each sample includes a single value or an n-tuple. The sample may represent a period of time of recorded cardiac activity (e.g., a cardiac EGM). Another sample may include other sensed data and/or information corresponding to physiological parameters.

Over time, the regulatory body of any jurisdiction may promulgate more laws and regulations for medical devices and the medical care being provided by such devices. This may necessitate multiple algorithms for classification of a particular malady type. Some of these algorithms, programmed into logic, may produce different health event detection results and still run a compatible interface to a same patient data configuration. Other algorithms are not compatible to each other for a number of reasons. For example, a particular algorithm may be compatible with an initial patient configuration and require the patient data in a specific structure/format for involving medical device functionality (i.e., health event detection); however, by modifying parameters of the initial patient data configuration used as input for the interface, two or more algorithms may have similar characteristics but require different interfaces for receiving input feeds of the patient data. The patient data may have to change its configuration to be compatible with the particular algorithm.

Multiple versions of a particular algorithm may develop over time, each algorithm being compatible to each other with comparable (but not same) functionality. In one example, the particular algorithm may implement a different (but still compatible) machine learning model for each algorithm version, and. The relevant regulatory body may be charged with validating, for medical device use, a pre-configured machine learning model and the particular machine learning algorithm for applying that model to determine a value for the specific variable. The relevant regulatory body may set forth one or more jurisdictional requirements or conditions for such validation.

The following description is in reference to medical system 2 of FIG. 1 Receiving, from a medical device such as IMD 10, a sequence of samples (e.g., as a service request) at computing system 20A may activate functionality of HMS 22, for example, to automatically configure the information in these samples into a suitable format for input to a validated algorithm. Having the samples properly configured into formatted patient data that is compatible with the validated algorithm enables an application of the algorithm without errors. In some examples, HMS 22 may verify that the requesting medical device operates in compliance with an applicable authority of the local jurisdiction or may reconfigure the samples generated and then, communicated by the medical device. In some examples, computing system 20A running HMS 22 for patients with cardiac maladies who use their medical devices to diagnose/detect, treat, and otherwise monitor patient activity may be configured to ensure that the medical device and any device components comply with applicable conditions of a relevant (e.g., government) authority (e.g., an agency similar to the Federal Drug Administration (FDA)). IMD 10 may combine an upload of recent patient data (as samples) with a request that identifies the selected algorithm as an appropriate algorithm for detecting cardiac events. Verification may involve determining that the selected algorithm is compatible with the samples of patient data.

HMS 22 may execute an example technique to determine whether using the selected algorithm (e.g., in a device of the cloud service or in IMD 10 itself) renders invalid for the local jurisdiction any medical device of patient 4. For example, when a user (e.g., patient 4 or clinician 8) selects a new algorithm for cardiac event detection, the user may submit a request to HMS 22 for verification that incorporating the new algorithm (into the device of the cloud service or in IMD 10 itself) does not invalidate a medical device and/or the new algorithm. As one example response, HMS 22 may determine that the new algorithm does not cause the medical device to violate any validation requirement and vice versa, that the medical device does not cause the new algorithm to violate any validation requirement. Based on that determination, HMS 22 may communicate an approval of the new algorithm for incorporation into the medical device. If, in the above example, HMS 22 determines that the new algorithm and/or the medical device violates the validation requirement and thereby, becomes invalid, HMS 22 may reply to the request with a denial of the new algorithm for incorporation into the medical device and/or for evaluation of patient data for occurrence(s) of a cardiac event.

The present disclosure further describes, as part of an automatic configuration (or re-configuration) of a classification of medical devices (e.g., cardiac monitors), HMS 22 (if needed) generates information describing ways to modify a configuration of that classification of medical devices. HMS 22 may generate information identifying certain medical device components as being compatible with a new algorithm, resulting in a properly configured medical device whose operations comply one or more (local) jurisdictional requirements.

Example 1. A medical system comprising: communication circuitry communicatively coupled to one or more devices comprising at least one of a medical device or another device of a patient; and processing circuitry configured to: access a data structure comprising an algorithm for health event detection in patient data generated by at least one of a medical device of the patient or a personal device of the patient based on a usage scenario, wherein association of the algorithm and the usage scenario in the data structure indicates that use of the algorithm for the usage scenario complies with one or more jurisdictional requirements; in response to the selection of the algorithm, generate information operative to perform an automatic configuration of the patient data to be interoperable with logic implementing the algorithm; and apply the selected algorithm to the automatically configured data.

Example 2. The medical system of Example 1, wherein the personal device comprises the input device, the output device, and the processing circuitry.

Example 3. The medical system of Example 1 or 2, wherein the processing circuitry is further configured to: based on a determination that the algorithm fails to comply with the one or more jurisdictional requirements, select, from a plurality of algorithms, a compatible algorithm for performing an application to the patient data in response to the algorithm failing to comply with the one or more jurisdictional requirements.

Example 4. The medical system of any of Examples 1-3, wherein the processing circuitry is further configured to: determine non-compliance of the algorithm based on a comparison, using a database system, between the algorithm and the one or more jurisdictional requirements, wherein the database system arranges respective datasets for the plurality of algorithms, wherein a dataset corresponding to the algorithm comprises approved usage scenario information for an application of the algorithm to the patient data, wherein the comparison results indicate a failure of the approved usage scenario information to satisfy the one or more jurisdictional requirements.

Example 5. The medical system of any of Examples 1-4, wherein the processing circuitry is further configured to: identify the compatible algorithm from plurality of algorithms based on a comparison, using a database system, between the compatible algorithm and the one or more jurisdictional requirements, wherein the database system arranges respective datasets for the plurality of algorithms, wherein a dataset corresponding to the compatible algorithm comprises approved usage scenario information for an application to the patient data, wherein the comparison results indicate satisfaction by the approved usage scenario information of the one or more jurisdictional requirements.

Example 6. The medical system of any of Examples 1-5, wherein the medical device is communicatively coupled to the personal device, wherein the medical device comprises at least one of an implantable medical device, a wearable medical device, a pacemaker/defibrillator, or a ventricular assist device (VAD) that comprises one or more sensors and sensing circuitry.

Example 7. A method for operating processing circuitry of a medical system comprising: responsive to receiving a service request for a medical device, determining data indicative of one or more jurisdictional requirements corresponding to a patient location, wherein the service request comprises data identifying an algorithm for health event detection in patient data generated by the medical device or another device; in accordance with the service request, accessing structured data comprising an approved usage scenario for the algorithm to determine whether the algorithm complies with the one or more jurisdictional requirements; based on a determination that the algorithm complies with the one or more jurisdictional requirements, automatically configuring the patient data to be interoperable with the algorithm; and generating health event detection results based on an application of the algorithm to the configured patient data.

Example 8. The method of Example 7 further comprising: based on a determination that the algorithm fails to comply with the one or more jurisdictional requirements, selecting, from a plurality of algorithms, a compatible algorithm for performing an application to the patient data in response to the algorithm failing to comply with the one or more jurisdictional requirements.

Example 9. The method of Example 7 or 8, further comprising: determining non-compliance of the algorithm based on a comparison, using a database system, between the algorithm and the one or more jurisdictional requirements, wherein the database system arranges respective datasets for the plurality of algorithms, wherein a dataset corresponding to the algorithm comprises approved usage scenario information for an application of the algorithm to the patient data, wherein the comparison results indicate a failure of the approved usage scenario information to satisfy the one or more jurisdictional requirements.

Example 10. The method of any of Examples 7-9, further comprising: identifying the compatible algorithm from plurality of algorithms based on a comparison, using a database system, between the compatible algorithm and the one or more jurisdictional requirements, wherein the database system arranges respective datasets for the plurality of algorithms, wherein a dataset corresponding to the compatible algorithm comprises approved usage scenario information for an application to the patient data, wherein the comparison results indicate satisfaction by the approved usage scenario information of the one or more jurisdictional requirements.

Example 11. The method of any of Examples 7-10, further comprising: identifying the compatible algorithm from plurality of algorithms based on a comparison, using a database system, between the compatible algorithm and the algorithm, wherein the database system arranges respective datasets for the plurality of algorithms, wherein a dataset corresponding to the compatible algorithm comprises a set of characteristics, wherein the comparison results indicate compatibility of the compatible algorithm.

Example 12. The method of any of Examples 7-11, wherein accessing the structured data comprises: generating information operative to implement the compatible algorithm in at least one of the medical device or the other device, wherein the generated information indicates at least one modification to device logic in memory of the at least one of the medical device or the other device, wherein the modified device logic is executed to apply the algorithm to the patient data.

Example 13. The method of any of Examples 7-12, wherein accessing the structured data comprises: accessing the structured data comprising an approved usage scenario for the algorithm for health event detection in the patient data generated by the at least one of the medical device of the patient or another device of the patient; in response to the selection of the algorithm, generating information operative to perform an automatic configuration of the patient data to be input for logic implementing the algorithm; and executing the logic for applying the selected algorithm to the automatically configured data.

Example 14. The method of any of Examples 7-13, wherein accessing the structured data comprises: accessing structured data comprising a usage scenario for an algorithm for health event detection in patient data generated by at least one of a medical device of the patient or another device of the patient, wherein association of the algorithm and the usage scenario in the structured data indicates that the usage scenario satisfies with one or more jurisdictional requirements; in response to the selection of the algorithm, generating information operative to perform an automatic configuration of the patient data to be interoperable for logic implementing the algorithm; and executing the logic for applying the selected algorithm to the automatically configured data.

Example 15. A method for operating processing circuitry of a medical system comprising: accessing structured data comprising a usage scenario for an algorithm for health event detection in patient data generated by at least one of a medical device or another device, wherein association of the algorithm and the usage scenario in the structured data indicates that the usage scenario satisfies with one or more jurisdictional requirements; responsive to receiving the patient data, generating information operative to perform an automatic configuration of the patient data to be interoperable for logic implementing the algorithm; and applying the selected algorithm to the automatically configured data.

Example 16. The method of Example 15, wherein a memory of the medical system comprises data configured to define the algorithm as a dataset comprising characteristics information and a unique identity amongst a plurality of algorithms.

Example 17. The method of Example 15 or 16, wherein generating the information comprises: determining updated parameter data that matches the characteristics information for the algorithm, the characteristics information comprising an input parameter or an output parameter.

Example 18. The method of any of Examples 15-17, wherein generating the information comprises: identifying parameter data of a logic component of the medical device or other device, wherein the parameter data defines a configuration of input data for the algorithm; and modifying the parameter data based on the characteristics information for the algorithm.

Example 19. The method of any of Examples 15-18, wherein generating the information comprises: generating instructions that a client application running on the other device executes to apply the at least one modification to the device logic.

Example 20. The method of any of Examples 15-19, wherein generating the information comprises: generating instructions that a client application running on the other device executes to load the logic implementing the algorithm into the device.

Example 21. The method of any of Examples 15-20, wherein the at least one of the medical device or the other device is configured with the logic implementing the algorithm and invokes the logic implementing the algorithm to monitor the patient data for patient activity indicative of health events.

Example 22. The method of any of Examples 15-21, wherein the at least one of the medical device or the other device is configured with the logic implementing the algorithm and invokes the logic to monitor the patient data for patient cardiac activity indicative of cardiac events.

Example 23. The method of any of Examples 15-22, wherein the at least one of the medical device or the other device is configured with the logic implementing the algorithm and invokes the logic to monitor the patient data for adjusting therapy delivery.

Example 24. The method of any of Examples 15-23, wherein the at least one of the medical device or the other device further invokes the logic to identify a modification to the therapy delivery in terms of a treatment amount, a treatment type, or a treatment schedule.

Example 25. The method of any of Examples 15-24, wherein the at least one of the medical device or the other device further invokes the logic to identify a treatment to remove, add, or modify for the therapy delivery.

Example 26. The method of any of Examples 15-25, wherein generating the information further comprises: generating information operative to perform an automatic configuration of the at least one of the medical device or the another device, wherein the generated information indicates at least one modification to device logic in memory of the at least one of the medical device or the other device, wherein the modified device logic configures the patient data to be interoperable with the logic implementing the algorithm.

Example 27. The method of any of Examples 15-26, wherein the usage scenario comprises attribution information indicative of a location corresponding to the patient.

Example 28. The method of any of Examples 15-27, wherein generating the information comprises: determining that the usage scenario comprises attribution information indicative of at least one of a country and a setting corresponding to a patient location satisfying the one or more jurisdictional requirements.

Example 29. The method of any of Examples 15-28, wherein the usage scenario comprises attribute information indicative of a clinical setting that overrides the one or more jurisdictional requirements.

Example 30. The method of any of Examples 15-29, wherein generating the information further comprises: determining that characteristics information of the algorithm satisfy the one or more jurisdictional requirements; and configuring the data structure to indicate that the algorithm satisfies the one or more jurisdictional requirements.

Example 31. The method of any of Examples 15-30, wherein generating the information further comprises: determining approval of the algorithm by a relevant authority corresponding to the patient location who promulgated the one or more jurisdictional requirements.

Example 32. A method performed by the medical system of any one or more of Examples 1-6.

Example 33. A method comprising any combination of the methods described herein.

Example 34. A medical system comprising processing circuitry configured to perform the method of any one or more of Examples 7-14.

Example 35. A medical system comprising processing circuitry configured to perform the method of any one or more of Examples 15-30.

Example 36. A non-transitory computer readable storage medium comprising program instructions configured to cause processing circuitry to perform the method of any one or more of Examples 7-14 or the method of any one or more of Examples 15-30.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

1. A medical system comprising:
communication circuitry communicatively coupled to one or more devices comprising at least one of a medical device or another device of a patient; and
processing circuitry configured to:
access, based on a usage scenario, a data structure comprising an algorithm for health event detection in patient data generated by at least one of the medical device or the other device of the patient, wherein association of the algorithm and the usage scenario in the data structure indicates that use of the algorithm for the usage scenario complies with one or more jurisdictional requirements;
in response to selection of the algorithm, generate information operative to perform an automatic configuration of the patient data to be interoperable with logic implementing the algorithm; and
apply the selected algorithm to the automatically configured data.

2. The medical system of claim 1, wherein the other device comprises an input device, an output device, and the processing circuitry.

3. The medical system of claim 1, wherein the processing circuitry is further configured to:
based on a determination that the algorithm fails to comply with the one or more jurisdictional requirements, select, from a plurality of algorithms, a compatible algorithm for performing an application to the patient data in response to the algorithm failing to comply with the one or more jurisdictional requirements.

4. The medical system of claim 1, wherein the processing circuitry is further configured to:
determine non-compliance of the algorithm based on a comparison, using a database system, between the algorithm and the one or more jurisdictional requirements, wherein the database system arranges respective datasets for the plurality of algorithms, wherein a dataset corresponding to the algorithm comprises approved usage scenario information for an application of the algorithm to the patient data, wherein the comparison results indicate a failure of the approved usage scenario information to satisfy the one or more jurisdictional requirements.

5. The medical system of claim 1, wherein the processing circuitry is further configured to:
identify the compatible algorithm from plurality of algorithms based on a comparison, using a database system, between the compatible algorithm and the one or more jurisdictional requirements, wherein the database system arranges respective datasets for the plurality of algorithms, wherein a dataset corresponding to the compatible algorithm comprises approved usage scenario information for an application to the patient data, wherein the comparison results indicate satisfaction by the approved usage scenario information of the one or more jurisdictional requirements.

6. The medical system of claim 1, wherein the medical device is communicatively coupled to the other device, wherein the medical device comprises at least one of an implantable medical device, a wearable medical device, a pacemaker/defibrillator, or a ventricular assist device (VAD) that comprises one or more sensors and sensing circuitry.

7. A method for operating processing circuitry of a medical system comprising:
responsive to receiving a service request for a medical device, determining data indicative of one or more jurisdictional requirements corresponding to a patient location, wherein the service request comprises data identifying an algorithm for health event detection in patient data generated by the medical device or another device;
in accordance with the service request, accessing structured data comprising an approved usage scenario for the algorithm to determine whether the algorithm complies with the one or more jurisdictional requirements;
based on a determination that the algorithm complies with the one or more jurisdictional requirements, automatically configuring the patient data to be interoperable with the algorithm; and
generating health event detection results based on an application of the algorithm to the configured patient data.

8. The method of claim 7 further comprising:
based on a determination that the algorithm fails to comply with the one or more jurisdictional requirements, selecting, from a plurality of algorithms, a compatible algorithm for performing an application to the patient data in response to the algorithm failing to comply with the one or more jurisdictional requirements.

9. The method of claim 7, further comprising:
determining non-compliance of the algorithm based on a comparison, using a database system, between the algorithm and the one or more jurisdictional requirements, wherein the database system arranges respective datasets for the plurality of algorithms, wherein a dataset corresponding to the algorithm comprises approved usage scenario information for an application of the algorithm to the patient data, wherein the comparison results indicate a failure of the approved usage scenario information to satisfy the one or more jurisdictional requirements.

10. The method of claim 7, further comprising:
identifying the compatible algorithm from plurality of algorithms based on a comparison, using a database system, between the compatible algorithm and the one or more jurisdictional requirements, wherein the database system arranges respective datasets for the plurality of algorithms, wherein a dataset corresponding to the compatible algorithm comprises approved usage scenario information for an application to the patient data, wherein the comparison results indicate satisfaction by the approved usage scenario information of the one or more jurisdictional requirements.

11. The method of claim 7, further comprising:
identifying the compatible algorithm from plurality of algorithms based on a comparison, using a database system, between the compatible algorithm and the algorithm, wherein the database system arranges respective datasets for the plurality of algorithms, wherein a dataset corresponding to the compatible algorithm comprises a set of characteristics, wherein the comparison results indicate compatibility of the compatible algorithm.

12. The method of any of claim 7, wherein accessing the structured data comprises:
generating information operative to implement the compatible algorithm in at least one of the medical device or the other device, wherein the generated information indicates at least one modification to device logic in memory of the at least one of the medical device or the other device, wherein the modified device logic is executed to apply the algorithm to the patient data.

13. The method of claim 7, wherein accessing the structured data comprises:
accessing the structured data comprising an approved usage scenario for the algorithm for health event detection in the patient data generated by the at least one of the medical device of the patient or another device of the patient;
in response to selection of the algorithm, generating information operative to perform an automatic configuration of the patient data to be input for logic implementing the algorithm; and
executing the logic for applying the selected algorithm to the automatically configured data.

14. The method of claim 7, wherein accessing the structured data comprises:
accessing structured data comprising a usage scenario for an algorithm for health event detection in patient data generated by at least one of a medical device of the patient or another device of the patient, wherein association of the algorithm and the usage scenario in the structured data indicates that the usage scenario satisfies with one or more jurisdictional requirements;
in response to selection of the algorithm, generating information operative to perform an automatic configuration of the patient data to be interoperable for logic implementing the algorithm; and
executing the logic for applying the selected algorithm to the automatically configured data.

15. A method for operating processing circuitry of a medical system comprising:
accessing structured data comprising a usage scenario for an algorithm for health event detection in patient data generated by at least one of a medical device or another device, wherein association of the algorithm and the usage scenario in the structured data indicates that the usage scenario satisfies with one or more jurisdictional requirements;
responsive to receiving the patient data, generating information operative to perform an automatic configuration of the patient data to be interoperable for logic implementing the algorithm; and
applying the algorithm to the automatically configured data.

16. The method of claim 15, wherein a memory of the medical system comprises data configured to define the algorithm as a dataset comprising characteristics information and a unique identity amongst a plurality of algorithms.

17. The method of claim 16, wherein generating the information comprises: determining updated parameter data that matches the characteristics information for the algorithm, the characteristics information comprising an input parameter or an output parameter.

18. The method of claim 16, wherein generating the information comprises:
identifying parameter data of a logic component of the medical device or other device, wherein the parameter data defines a configuration of input data for the algorithm; and
modifying the parameter data based on the characteristics information for the algorithm.

19. The method of claim 18, wherein generating the information comprises:
generating instructions that a client application running on the other device executes to apply the at least one modification to the device logic.

20. The method of claim 18, wherein the at least one of the medical device or the other device is configured with the logic implementing the algorithm and invokes the logic implementing the algorithm to monitor the patient data for patient activity indicative of health events.
